SEQUENCE LISTING (17 sequences)

SEQ ID NO: 1 - protein, 317 aa, artificial sequence
  Artificial Peptide
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVDWLLTVPLLLVQTVAVIERYVVVCKPMSNFRFGENHPLLIKLTVASVLMIATGYPGEISDDITTRIIWGTVSTIPFAYILYVLWVELSFTVKEAAAQQQESATTQVQTLVRNMRWLLLLSWGVYPIAYLLPMLGVSGTSAAVGVQVGYTIADVLAKPVFGLLVFAIALVKTKADQESSEPHAAIGAAANKSGGSLIS

SEQ ID NO: 2 - protein, 344 aa, artificial sequence
  Artificial Peptide
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVDWLLTVPLLLVQTVAVIERYVVVCKPMSNFRFGENHPLLIKLTVASVLMIATGYPGEISDDITTRIIWGTVSTIPFAYILYVLWVELSFTVKEAAAQQQESATTQVQTLVRNMRWLLLLSWGVYPIAYLLPMLGVSGTSAAVGVQVGYTIADVLAKPVFGLLVFAIALVKTKADQESSEPHAAIGAAANKSGGSLISGSASASNGASDYKDDDDKEFFCYENEV

SEQ ID NO: 3 - protein, 344 aa, artificial sequence
  Artificial Peptide
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVDWLLTVPLLLVETVAVIERYVVVCKPMSNFRFGENHPLLIKLTVASVLMIATGYPGEISDDITTRIIWGTVSTIPFAYILYVLWVELSFTVKEAAAQQQESATTQVQTLVRNMRWLLLLSWGVYPIAYLLPMLGVSGTSAAVGVQVGYTIADVLAKPVFGLLVFAIALVKTKADQESSEPHAAIGAAANKSGGSLISGSASASNGASDYKDDDDKEFFCYENEV

SEQ ID NO: 4 - protein, 311 aa, artificial sequence
  Artificial Peptide
MASQVVYGEWASTHTECYNMSRIDSTFVSLLQLVWAVVSGCQTIFMISRAPKVPWESVYLPFVESITYALASTGNGTLQMRDGRFFPWSRMASWLCTCPIMLGQISNMIERYVVVCKPMSNFRFGENHIPLNPIAQAASIIRVVMGITATISPAEYMKWLFFFFGATCLVFEYSVVFTIFQVGLFTVKEAAAQQQESATTQAQKVVVRIKMLRLIFFIAWTMFPIVWLISPTGVCVIHENVSAILYLLADGLCKNTYGVILWSTAWGVLEGKWDPACLPGQEKPEADDPFGLNHEKNAPPNDEVNIRMFGR

SEQ ID NO: 5 - protein, 11 aa, artificial sequence
  Artificial Peptide
IERYVVVCKPM

SEQ ID NO: 6 - protein, 20 aa, artificial sequence
  Artificial Peptide
IERYVVVCKPMSNFRFGENH

SEQ ID NO: 7 - protein, 17 aa, artificial sequence
  Artificial Peptide
FTVKEAAAQQQESATTQ

SEQ ID NO: 8 - protein, 298 aa, Gloeobacter violaceus
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVDWLLTVPLLLVETVAVLTLPAKEARPLLIKLTVASVLMIATGYPGEISDDITTRIIWGTVSTIPFAYILYVLWVELSRSLVRQPAAVQTLVRNMRWLLLLSWGVYPIAYLLPMLGVSGTSAAVGVQVGYTIADVLAKPVFGLLVFAIALVKTKADQESSEPHAAIGAAANKSGGSLIS

SEQ ID NO: 9 - protein, 348 aa, Bos taurus
MNGTEGPNFYVPFSNKTGVVRSPFEAPQYYLAEPWQFSMLAAYMFLLIMLGFPINFLTLYVTVQHKKLRTPLNYILLNLAVADLFMVFGGFTTTLYTSLHGYFVFGPTGCNLEGFFATLGGEIALWSLVVLAIERYVVVCKPMSNFRFGENHAIMGVAFTWVMALACAAPPLVGWSRYIPEGMQCSCGIDYYTPHEETNNESFVIYMFVVHFIIPLIVIFFCYGQLVFTVKEAAAQQQESATTQKAEKEVTRMVIIMVIAFLICWLPYAGVAFYIFTHQGSDFGPIFMTIPAFFAKTSAVYNPVIYIMMNKQFRNCMVTTLCCGKNPLGDDEASTTVSKTETSQVAPA

SEQ ID NO: 10 - DNA, 1036 bp, artificial sequence
  Artificial Nucleic Acid
ggagacccaagctggctagcgtttaaacttaagcttgccaccatgttgatgaccgtattttcttctgcacctgaacttgcccttctcggatcaacctttgcccaggtcgatccttcaaacttatcggtctcagattcgctgacctatggtcagttcaatctggtttacaacgctttctcgtttgccatcgcggcaatgttcgcatctgccctcttcttcttcagcgctcaggcactcgtcggtcaacgataccggttggccttgcttgtttcagcaattgttgtgagtatcgctgggtaccactactttcggatcttcaatagttgggatgctgcctacgttctggagaatggcgtgtattccctgactagcgaaaaattcaacgacgcctaccgctatgtggattggctgttgaccgtgcctctgttgctggtgcaaacagtggcagtgatcgagcggtacgtagtggtgtgcaagcccatgagcaacttccgcttcggggagaaccaccccttgctgatcaaactgacggtggcttcagttctgatgattgccacgggctaccccggcgagatttctgacgacattacgactcgcatcatctggggtacggtcagcacgattcccttcgcctacatcctctatgtgttgtgggtcgaactgtccttcaccgtcaaggaggctgcagcccagcagcaggagtcggccaccactcaggtacaaaccctggtccgcaacatgcggtggctgctgttgctctcctggggtgtttacccgatcgcataccttctacccatgcttggagtatccggtacgtccgcggctgtcggcgttcaggttggctatacgatcgcagacgtgctggcgaagcctgtatttggtcttctagtcttcgcgattgcactcgtgaaaacaaaagcagatcaagaaagcagtgaaccacatgccgcaataggtgctgctgcaaataaatcgggaggcagtcttatctcctgatctagagggcccgtttaaacccgctgatcagcctcgactg

SEQ ID NO: 11 - DNA, 33 bp, artificial sequence
  Artificial Nucleic Acid
atcgagcggtacgtagtggtgtgcaagcccatg

SEQ ID NO: 12 - DNA, 60 bp, artificial sequence
  Artificial Nucleic Acid
atcgagcggtacgtagtggtgtgcaagcccatgagcaacttccgcttcggggagaaccac

SEQ ID NO: 13 - DNA, 51 bp, artificial sequence
  Artificial Nucleic Acid
ttcaccgtcaaggaggctgcagcccagcagcaggagtcggccaccactcag

SEQ ID NO: 14 - protein, 348 aa, Homo sapiens [sequence omitted]

SEQ ID NO: 15 - DNA, 1047 bp, Homo sapiens
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa

SEQ ID NO: 16 - DNA, 1047 bp, Bos taurus
atgaacgggaccgagggcccaaacttctacgtgcctttctccaacaagacgggcgtggtgcgcagccccttcgaggccccgcagtactacctggcggagccatggcagttctccatgctggccgcctacatgttcctgctgatcatgcttggcttccccatcaacttcctcacgctgtacgtcacagtccagcacaagaagctgcgcacacccctcaactacatcctgctcaacctggccgtggccgacctcttcatggtcttcgggggcttcaccaccaccctctacacctctctgcacggatacttcgtctttgggcccacgggctgcaacctggagggcttctttgccaccctgggcggtgaaattgcactgtggtccttggtggtcctggccatcgagcggtacgtggtggtgtgcaagcccatgagcaacttccgcttcggggagaaccacgccatcatgggcgtcgccttcacctgggtcatggctctggcctgtgccgcgccccccctcgtcggctggtccaggtacatcccggagggcatgcagtgctcgtgcgggattgactactacacgccccacgaggagaccaacaatgagtcgttcgtcatctacatgttcgtggtccacttcatcatccccctgattgtcatattcttctgctacgggcagctggtgttcaccgtcaaggaggcggctgcccagcagcaggagtcggccaccactcagaaggccgagaaggaggtcacccgcatggtgatcatcatggtcatcgctttcctaatctgctggctgccctacgctggggtggcgttctacatcttcacccatcagggctctgactttggccccatcttcatgaccatcccggctttctttgccaagacttctgccgtctacaaccccgtcatctacatcatgatgaacaagcagttccggaactgcatggtcaccactctctgctgtggcaagaacccgctgggtgacgacgaggcctccaccaccgtctccaagacagagaccagccaggtggcgcctgcctaa

SEQ ID NO: 17 - DNA, 897 bp, Gloeobacter violaceus
atgttgatgaccgtattttcttctgcacctgaacttgcccttctcggatcaacctttgcccaggtcgatccttcaaacttatcggtctcagattcgctgacctatggtcagttcaatctggtttacaacgctttctcgtttgccatcgcggcaatgttcgcatctgccctcttcttcttcagcgctcaggcactcgtcggtcaacgataccggttggccttgcttgtttcagcaattgttgtgagtatcgctgggtaccactactttcggatcttcaatagttgggatgctgcctacgttctggagaatggcgtgtattccctgactagcgaaaaattcaacgacgcctaccgctatgtggattggctgttgaccgtgcctctgttgctggtggagacagtggcagtgctgacgttgcctgcaaaggaggcaagacccttgctgatcaaactgacggtggcttcagttctgatgattgccacgggctaccccggcgagatttctgacgacattacgactcgcatcatctggggtacggtcagcacgattcccttcgcctacatcctctatgtgttgtgggtcgaactgtccaggtcccttgtccgccagcccgctgctgtacaaaccctggtccgcaacatgcggtggctgctgttgctctcctggggtgtttacccgatcgcataccttctacccatgcttggagtatccggtacgtccgcggctgtcggcgttcaggttggctatacgatcgcagacgtgctggcgaagcctgtatttggtcttctagtcttcgcgattgcactcgtgaaaacaaaagcagatcaagaaagcagtgaaccacatgccgcaataggtgctgctgcaaataaatcgggaggcagtcttatctcctag

(19) United States
(12) Patent Application Publication
(10) Pub. No.: US 2022/0025018 A1
(43) Pub. Date: Jan. 27, 2022

(54) PREVENTING, AND SUPPRESSING PROGRESSION OF, RETINAL DISEASE, IMPROVING VISUAL COGNITIVE BEHAVIORAL FUNCTION, AND STRENGTHENING VISUAL FUNCTION

(71) Applicant: Restore Vision Inc., Tokyo (JP)

(72) Inventors: Toshihide Kurihara, Tokyo (JP); Yusaku Katada, Tokyo (JP); Kazuo Tsubota, Tokyo (JP)

(21) Appl. No.: 17/424,144

(22) PCT Filed: Jan. 18, 2019
(86) PCT No.: PCT/JP2019/001565

§ 371 Date: Jul. 19, 2021

(51) Int. Cl. C07K 14/72; A61P 27/02

(52) U.S. Cl. CPC C07K 14/723; A61K 38/00; A61P 27/02

(57) ABSTRACT

Provided are a chimeric protein of two types of rhodopsins, an ion-transporting rhodopsin and a G protein-coupled receptor rhodopsin, and a nucleic acid encoding the same, for the prevention and suppression of progression of retinal diseases, the improvement in visual cognitive behavioral functions (e.g., improvement in light-dark determination functions, improvement in bright spot evading functions, and/or crisis avoidance functions), and the enhancement of visual functions (e.g., improvement in visual acuity). The present invention also provides a method for preventing or suppressing the progression of a disease, disorder or symptom of the retina, for improving a visual cognitive behavioral function (e.g., improvement in light-dark determination functions, improvement in bright spot evading functions, and/or crisis avoidance functions), or for enhancing visual functions (e.g., improvement in visual acuity), in a subject, where the method comprises the step of administering an effective amount of a nucleic acid encoding a chimeric protein of an ion-transporting receptor rhodopsin and a G protein-coupled receptor rhodopsin to the subject.

### TECHNICAL FIELD

The present invention relates to prevention and suppression of progression of retinal diseases, improvement in visual cognitive behavioral functions, and enhancement of visual functions.

### BACKGROUND ART

Rhodopsin is a photosensitive receptor with a seven-time-transmembrane structure in the retina of humans and animals, and rhodopsin is also applied in medicine.

### SUMMARY OF INVENTION

### Solution to Problem

The inventors have found that a chimeric protein of two types of rhodopsins, an ion-transporting rhodopsin and a G protein-coupled receptor rhodopsin, has effects for the prevention and suppression of progression of retinal diseases, the improvement in visual cognitive behavioral functions, and the enhancement of visual functions, thereby completing the present invention.

The present invention provides the following:

(Item 1) A composition for preventing or suppressing the progression of a disease, disorder or symptom of the retina, the composition comprising a nucleic acid encoding a chimeric protein of an ion-transporting receptor rhodopsin and a G protein-coupled receptor rhodopsin.
(Item 2) A composition for improving a visual cognitive behavioral function (e.g., improvement in light-dark determination functions, improvement in bright spot evading functions, and/or crisis avoidance functions), the composition comprising a nucleic acid encoding a chimeric protein of an ion-transporting receptor rhodopsin and a G protein-coupled receptor rhodopsin.
(Item 3) A composition for enhancing a visual function (e.g., improvement in visual acuity), the composition comprising a nucleic acid encoding a chimeric protein of an ion-transporting receptor rhodopsin and a G protein-coupled receptor rhodopsin.
(Item 4) The composition of Item 1, wherein the disease, disorder or symptom includes retinal degenerative disease.
(Item 5) The composition of Item 1 or 4, wherein the disease, disorder or symptom is retinitis pigmentosa.
(Item 6) The composition of any one of Items 1, 4 and 5, wherein the disease, disorder or symptom is autosomal dominantly inherited.
(Item 7) The composition of any one of Items 1 and 4 to 6, wherein the composition is for preventing or suppressing the progress of retinitis pigmentosa.
(Item 8) The composition of any one of Items 1 and 4 to 7, characterized in that the composition is administered to a subject before the onset or immediately after the onset of the disease, disorder or symptom.
(Item 9) The composition of any one of Items 1 to 8, characterized in that the composition is administered once.
(Item 10) The composition of any one of Items 1 to 9, characterized by being administered at a unit dose of 0.1×10¹¹ to 10×10¹¹ vg/eye.
(Item 11) The composition of any one of Items 1 to 10, wherein, of base sequences encoding the ion-transporting receptor rhodopsin, a base sequence encoding a second loop on a cytoplasmic side and/or a third loop on a cytoplasmic side is substituted by a base sequence encoding a second loop on a cytoplasmic side and/or a third loop on a cytoplasmic side of the G protein-coupled receptor rhodopsin.
(Item 12) The composition of any one of Items 1 to 11, wherein the ion-transporting receptor rhodopsin is derived from cyanobacteria (blue-green bacteria).
(Item 13) The composition of any one of Items 1 to 12, wherein the G protein-coupled receptor rhodopsin is derived from a mammal.
(Item 14) The composition of any one of Items 1 to 13, wherein the chimeric protein has an amino acid sequence in which glutamic acid, corresponding to position 132 of the amino acid sequence of SEQ ID NO: 8, is substituted by glutamine.
(Item 15) The composition of any one of Items 1 to 14,

wherein the chimeric protein has any one of the following:

(a) an amino acid sequence set forth in SEQ ID NOs: 1-4 or a fragment thereof;
(b) an amino acid sequence having at least 80% identity to (a); and
(c) an amino acid sequence with one or more amino acids substituted, added and/or deleted with respect to (a) or (b), and the chimeric protein also has biological activity, or

wherein the nucleic acid encoding the chimeric protein has any one of the following:

(A) a base sequence encoding an amino acid sequence set forth in any of SEQ ID NOs: 1-4, or a base sequence set forth in SEQ ID NO: 10, or a fragment thereof;
(B) a nucleic acid having at least 80% identity to (A);
(C) a nucleic acid with one or more nucleotides substituted, added and/or deleted with respect to (A) or (B); and
(D) a nucleic acid that hybridizes to any of (A) to (C) under stringent conditions, and the chimeric protein has biological activity.
(Item 16) The composition of any one of Items 1 to 15, wherein the base sequence is included in a vector.
(Item 17) A composition for preventing or suppressing the progression of a disease, disorder or symptom of the retina, the composition comprising a chimeric protein of an ion-transporting receptor rhodopsin and a G protein-coupled receptor rhodopsin.
(Item 18) A composition for improving a visual cognitive behavioral function (e.g., improvement in light-dark determination functions, improvement in bright spot evading functions, and/or crisis avoidance functions), the composition comprising a chimeric protein of an ion-transporting receptor rhodopsin and a G protein-coupled receptor rhodopsin.
(Item 19) A composition for enhancing a visual function (e.g., improvement in visual acuity), the composition comprising a chimeric protein of an ion-transporting receptor rhodopsin and a G protein-coupled receptor rhodopsin.
(Item 20) The composition of any one of Items 17 to 19, further having any characteristic in any one or more of Items 4 to 16.
(Item 21) A nucleic acid encoding a chimeric protein of an ion-transporting receptor rhodopsin and a G protein-coupled receptor rhodopsin, for preventing or suppressing the progression of a disease, disorder or symptom of the retina.
(Item 22) A nucleic acid encoding a chimeric protein of an ion-transporting receptor rhodopsin and a G protein-coupled receptor rhodopsin, for improving a visual cognitive behavioral function (e.g., improvement in light-dark determination functions, improvement in bright spot evading functions, and/or crisis avoidance functions).
(Item 23) A nucleic acid encoding a chimeric protein of an ion-transporting receptor rhodopsin and a G protein-coupled receptor rhodopsin, for enhancing a visual function (e.g., improvement in visual acuity).
(Item 24) A chimeric protein of an ion-transporting receptor rhodopsin and a G protein-coupled receptor rhodopsin, for preventing or suppressing the progression of a disease, disorder or symptom of the retina.
(Item 25) A chimeric protein of an ion-transporting receptor rhodopsin and a G protein-coupled receptor rhodopsin, for improving a visual cognitive behavioral function (e.g., improvement in light-dark determination functions, improvement in bright spot evading functions, and/or crisis avoidance functions).
(Item 26) A chimeric protein of an ion-transporting receptor rhodopsin and a G protein-coupled receptor rhodopsin, for enhancing a visual function (e.g., improvement in visual acuity).
(Item 27) The nucleic acid or protein of any one of Items 21 to 26, further having any characteristic in any one or more of Items 4 to 16.
(Item 28) A method for preventing or suppressing the progression of a disease, disorder or symptom of the retina in a subject, the method comprising: administering an effective amount of a nucleic acid encoding a chimeric protein of an ion-transporting receptor rhodopsin and a G protein-coupled receptor rhodopsin to the subject.
(Item 29) A method for improving a visual cognitive behavioral function (e.g., improvement in light-dark determination functions, improvement in bright spot evading functions, and/or crisis avoidance functions) in a subject, the method comprising: administering an effective amount of a nucleic acid encoding a chimeric protein of an ion-transporting receptor rhodopsin and a G protein-coupled receptor rhodopsin to the subject.
(Item 30) A method for enhancing a visual function (e.g., improvement in visual acuity) in a subject, the method comprising: administering an effective amount of a nucleic acid encoding a chimeric protein of an ion-transporting receptor rhodopsin and a G protein-coupled receptor rhodopsin to the subject.
(Item 31) A method for preventing or suppressing the progression of a disease, disorder or symptom of the retina in a subject, the method comprising: administering an effective amount of a chimeric protein of an ion-transporting receptor rhodopsin and a G protein-coupled receptor rhodopsin to the subject.
(Item 32) A method for improving a visual cognitive behavioral function (e.g., improvement in light-dark determination functions, improvement in bright spot evading functions, and/or crisis avoidance functions) in a subject, the method comprising: administering an effective amount of a chimeric protein of an ion-transporting receptor rhodopsin and a G protein-coupled receptor rhodopsin to the subject.
(Item 33) A method for enhancing a visual function (e.g., improvement in visual acuity) in a subject, the method comprising: administering an effective amount of a chimeric protein of an ion-transporting receptor rhodopsin and a G protein-coupled receptor rhodopsin to the subject.
(Item 34) The method of any one of Items 28 to 33, further having any characteristic in any one or more of Items 4 to 16.
(Item 35) Use of a nucleic acid encoding a chimeric protein of an ion-transporting receptor rhodopsin and a G protein-coupled receptor rhodopsin, in the manufacture of a pharmaceutical for preventing or suppressing the progression of a disease, disorder or symptom of the retina.
(Item 36) Use of a nucleic acid encoding a chimeric protein of an ion-transporting receptor rhodopsin and a G protein-coupled receptor rhodopsin, in the manufacture of a pharmaceutical for improving a visual cognitive behavioral function (e.g., improvement in light-dark determination functions, improvement in bright spot evading functions, and/or crisis avoidance functions).
(Item 37) Use of a nucleic acid encoding a chimeric protein of an ion-transporting receptor rhodopsin and a G protein-coupled receptor rhodopsin, in the manufacture of a pharmaceutical for enhancing a visual function (e.g., improvement in visual acuity).
(Item 38) Use of a chimeric protein of an ion-transporting receptor rhodopsin and a G protein-coupled receptor rhodopsin, in the manufacture of a pharmaceutical for preventing or suppressing the progression of a disease, disorder or symptom of the retina.
(Item 39) Use of a chimeric protein of an ion-transporting receptor rhodopsin and a G protein-coupled receptor rhodopsin, in the manufacture of a pharmaceutical for improving a visual cognitive behavioral function (e.g., improvement in light-dark determination functions, improvement in bright spot evading functions, and/or crisis avoidance functions).
(Item 40) Use of a chimeric protein of an ion-transporting receptor rhodopsin and a G protein-coupled receptor rhodopsin, in the manufacture of a pharmaceutical for enhancing a visual function (e.g., improvement in visual acuity).
(Item 41) The use of any one of Items 35 to 40, further having any characteristic in any one or more of Items 4 to 16.
(Item 42) A nucleic acid having any one of the following:
(a) a nucleic acid having an amino acid sequence set forth in SEQ ID NO: 10 or a fragment thereof;
(b) a nucleic acid having at least 80% identity to (a);
(c) a nucleic acid with one or more nucleotides substituted, added and/or deleted with respect to (a) or (b); and
(d) a nucleic acid that hybridizes to any of (a) to (c) under stringent conditions,

wherein a protein encoded by the nucleic acid has biological activity.

In the present invention, it is intended that the above one or more features may be provided in further combinations, in addition to the explicit combinations. Still further embodiments and advantages of the present invention will be appreciated by those skilled in the art upon reading and understanding the following detailed description as necessary.

### Advantageous Effects of Invention

The present invention provides preventive and progression-suppressing effects for diseases, disorders or symptoms of the retina. The present invention also provides effects for improving visual cognitive behavioral functions (e.g., improvement in light-dark determination functions, improvement in bright spot evading functions, and/or crisis avoidance functions). The present invention further provides effects for augmenting visual functions, such as improvement in visual acuity.

### BRIEF DESCRIPTION OF DRAWINGS

FIG. 1 shows experimental results of thinning of photoreceptor cells. The upper panel shows a tomographic photograph of the retina, and the arrows indicate the photoreceptor layer. Scale bar: 50 μm. The lower panel shows the results of quantitative comparison of photoreceptor layer thickness by OCT. The upper left is the tomographic photograph of the 24th day, while the upper right is the tomographic photograph of the 31st day. The lower left shows the contrast between the control and the chimera on the 24th day, while the lower right shows the contrast between the control and the chimera on the 31st day.

FIG. 2 shows progression suppressing effects after the introduction of chimeric rhodopsin. The upper left panel shows representative waveforms of mixed ERG for chimeric-rhodopsin-gene-transfected treatment eyes and the control. The lower left, upper right, and lower right show quantitative comparisons of the b-wave amplitudes of the mixed response, rod response, and cone response, respectively.

FIG. 3 shows results of a light-dark discrimination reaction test (light-dark selection box test=LDT). The upper panel is a schematic diagram. Below is a comparison of mouse dwell times in the bright compartment. From the left, shown are healthy mice (B6), blind mice (rd1) and treated mice. The symbol, “*”, Indicates statistical significance (p<0.01).

FIG. 4 shows results of demonstration of enhancement of visual functions. The upper left is a schematic photograph. The lower panel shows the visual evoked potential (VEP). The control is shown on the left, while the treated group is shown in the middle. The lower right shows the potential contrast, where a significant increase in the amplitude was observed in the chimeric-treated mice (50.0±3.49 μV) with respect to the control (35.12±3.90 μV).

### DESCRIPTION OF EMBODIMENTS

Hereinafter, the present invention will be described while showing the best mode. Throughout the present specification, it should be understood that the representation of a singular form also includes the concept of a plural form thereof, unless otherwise stated. It should thus be understood that singular articles (e.g., “a”, “an”, “the”, etc. in the English language) also include the concept of a plural form thereof, unless otherwise stated. It should also be understood that the terms used herein are used in the meaning commonly used in the art, unless otherwise stated. Thus, unless otherwise defined, all technical terms and scientific terms used herein have the same meaning as commonly understood by those skilled in the art to which the present invention pertains. In case of conflict, the present specification (including definitions) takes precedence.

### Definition

The definitions and/or basic technical contents of terms particularly used in the present specification will be described below as appropriate.

As used herein, a “rhodopsin” is a protein having a pigment called retinal inside, which is activated by receiving light, thereby transmitting a visual signal to the brain. Ion-transporting receptor rhodopsins, typified by those of microbial origin, can be repeatedly activated by absorbing light because they do not release retinal by light irradiation; however, they are unable to activate a G protein like the G protein-coupled receptor rhodopsins as typified by those of animal origin. In contrast, the chimeric rhodopsin with an ion-transporting receptor rhodopsin and a G protein-coupled receptor rhodopsin, as provided in the present disclosure, is thought to have enhanced functions compared to the conventional rhodopsin. In particular, the ion-transporting receptor rhodopsin can preferably be of microbial origin, and those that can be repeatedly used are utilized. Furthermore, when the G protein-coupled receptor rhodopsin of animal origin, preferably of mammalian origin, is utilized, high activity via an endogenous G protein can be obtained while the function of repeated activation is retained. Without wishing to be bound by theory, the chimeric protein utilized in the present invention is expressed in mammals, such as rodents and primates, while retaining sufficient activity, as demonstrated by the animal models; thus, the chimeric protein is capable of achieving preventive and progression-suppressing effects for diseases, disorders or symptoms of the retina, and in particular, the prevention or suppression of progression of retinitis pigmentosa, or providing improvement in visual cognitive behavioral functions (e.g., improvement in light-dark determination functions, improvement in bright spot evading functions, and/or crisis avoidance functions), or exerting effects for augmenting visual functions, such as improvement in visual acuity. As such, the chimeric protein obtained by combining different types of receptors with completely different functions has been actually found to function in these various applications. In the present specification, it has been confirmed that in a model system in which the onset rate is slow and the preventive effect and suppression of progression can be observed, the onset and progression of the disease can be suppressed by the administration thereof in the state before the actual blindness, where the suppression of progression of retinal diseases such as retinitis pigmentosa has been achieved.

As used herein, an “ion-transporting receptor rhodopsin” refers to any rhodopsin having a function of transporting ions, and examples thereof include an ion pumping receptor rhodopsin and an ion channeling receptor rhodopsin.

With regard to the ion-transporting receptor rhodopsin, the conformational compatibility and the membrane transfer efficiency with the G protein activation loop are considered to be important. In particular, the ion-transporting receptor rhodopsins of microbial origin have good conformational compatibility and membrane transfer efficiency with the G protein activation loop, and among them, those pertaining to the genus Gloeobacter are preferable. In particular, among the microorganisms pertaining to the genus Gloeobacter, Gloeobacter violaceus is preferable. It is also preferable to combine and utilize the rhodopsin (e.g., SEQ ID NO: 8) of microorganisms pertaining to the genus Gloeobacter with a G protein-coupled receptor rhodopsin of mammalian origin, and preferably a G protein-coupled receptor rhodopsin of Artiodactyla, such as cow (e.g., SEQ ID NO: 9), or primates such as humans (e.g., SEQ ID NO: 14 and SEQ ID NO: 15), among the G protein-coupled receptor rhodopsins of animal origin. The genus Gloeobacter is also preferable in terms of having an important property of being expressed well in E. coli, which is an eubacterium, and human cells, which are eukaryotes.

As used herein, a “G protein-coupled receptor rhodopsin” refers to a rhodopsin classified as a G protein-coupled receptor, which is a type of receptor existing on the cytoplasmic membrane of eukaryotic cells or on the constituent membrane inside the cell. The G protein-coupled receptor is said to have seven α-helix structures that penetrate the cytoplasmic membrane, with the N-terminal side being extracellular and the C-terminal side being intracellular, and three extracellular loops (ECL1/2/3) and three intracellular loops (ICL1/2/3). The rhodopsin is composed of apoprotein and chromophore retinal, and retinal absorbs light to isomerize and cause structural changes in the protein part, driving the intracellular signal transduction system via the G protein.

As used herein, a “disease, disorder or symptom of the retina” refers to any disease, disorder or symptom related to the retina, and the examples include retinal degenerative diseases (retinitis pigmentosa, age-related macular degeneration, etc.), retinopathy (e.g., diabetic retinopathy, proliferative retinopathy, simple retinopathy, etc.), floater, retinal tear, retinal detachment (e.g., rhegmatogenous retinal detachment, non-rhegmatogenous retinal detachment, etc.), and the like. Herein, the present invention is capable of preventing, treating or suppressing the progression of retinal degenerative diseases, age-related macular degeneration, myopic maculopathy, macular dystrophy, diabetic retinopathy, uveitis, retinal detachment, and the like. Examples of the disorder or symptom include disorders in visual acuity, contrast sensitivity, light-dark adaptation, color vision, etc., and symptoms associated therewith.

As used herein, a “visual cognitive behavioral function” refers to functioning of the visual information recognized by the visual organs (eyes, etc.) as the behavior of the target organism, where the visual cognitive behavioral function appears as actual behaviors, such as light-dark determination functions, bright spot evading functions and crisis avoidance functions. The visual cognitive behavioral function is such a function that can be confirmed, not only by confirming photosensitivity, but also by actually verifying it with an animal model (see Example 2).

As used herein, a “light-dark determination function” refers to an ability or function that can judge light and dark. The improvement therein may be any improvement in the light-dark determination function, the improvement of which also encompasses, for example, improvement in being able to determine what could not be determined as light or dark, and improvement in matters in which the difference between light and dark can be barely recognized.

As used herein, a “bright spot evading function” refers to the ability or function to move away from a light source or avoid bright light. The improvement therein refers to restoration or enhancement of the ability to avoid a bright spot.

As used herein, a “crisis avoidance function” refers to a function or ability to avoid a crisis based on a visual function. The improvement therein encompasses regenerating crisis avoidance ability, and additionally, raising the levels thereof.

As used herein, the “enhancement” or “augmentation” of the “visual function” refers to improvement, enhancement or augmentation of any visual functions (e.g., visual acuity, color vision, contrast sensitivity, light-dark adaptation, etc.).

As used herein, an “improvement in visual acuity” refers to improving or recovering the visual acuity. In the case of humans, for example, the visual acuity can be measured by a Snellen chart or an E chart in addition to a visual acuity test using a Randold ring, and can be expressed by decimal visual acuity or fractional visual acuity. These can also be displayed with log MAR visual acuity. In the case of mice, the visual acuity can be measured using visual stimuli that manipulate the spatial frequencies of light and dark stripes. The visual acuity can also be determined experimentally by measuring the visual evoked potential.

As used herein, a “retinal degenerative disease” refers to any disease caused by degeneration of the retina, and examples thereof include, for example, retinitis pigmentosa, age-related macular degeneration, and the like.

As used herein, “retinitis pigmentosa” is a hereditary disease with abnormalities in the retina, in which the photoreceptor and pigment epithelial cells of the retina are extensively degenerated. In the retinitis pigmentosa, three symptoms appear: night blindness (difficulty seeing things in the dark), narrowing of the visual field (narrow vision), and decreased visual acuity. The degeneration of only rod cells among the photoreceptor cells is called rod dystrophy, while the degeneration of both rod cells and cone cells, among the photoreceptor cells, is called rod cone dystrophy. Studies are being promoted on gene therapy, artificial retina, retinal restoration, photoreceptor protection therapy, etc., but no cure has been established for these diseases. Since these diseases are binocularly progressive and often lead to social blindness in the 40s at the earliest, it is very significant to suppress their progression.

As used herein, the “retinitis pigmentosa” includes autosomal recessive inherited retinitis pigmentosa as well as autosomal dominant inherited retinitis pigmentosa and X-chromosome recessive inherited retinitis pigmentosa. The most common retinitis pigmentosa is the type showing autosomal recessive inheritance, which accounts for about 35% of the total. The next most common is the type showing autosomal dominant inheritance, which accounts for 10% of the total. The least common is the type showing X-linked inheritance (X-chromosome recessive inheritance), which accounts for about 5% of the total. It should be noted as a remarkable point, in particular, that rhodopsin was also able to suppress the progression of autosomal dominant retinitis pigmentosa. The autosomal dominant retinitis pigmentosa is mainly caused by periferin (PRPH2, also known as RDS), in addition to rhodopsin abnormalities. As for the autosomal recessive inheritance, known are EYS, rod cGMP-phosphodiesterase α and β subunits, rod cyclic nucleotide-sensitive cation channels, retinal guanyl cyclase, RPE65, Cellular retinyl aldehyde binding protein, arrestin, usherin (USH2), and other genes. As for the X-linked retinitis pigmentosa, examples thereof include a retinitis pigmentosa GTPase regulator (RPGR), RP2 and the like.

As used herein, “suppression of progression” refers to the suppression of progression of a disease (e.g., retinitis pigmentosa), where the suppression encompasses a reduction in the rate of exacerbations compared to the absence of treatment, as well as maintenance and improvement in the disease levels. If a certain disease has not developed, it falls under “prevention of onset”. As used herein, the “onset” refers to appearance of a subjective symptom of disease from a state in which no such subjective symptom of the disease appears. Examples of the subjective symptoms include symptoms such as night blindness, narrowing of vision, photophobia, decreased visual acuity and defective color vision.

As used herein, “immediately after” the “onset” refers to within a certain period of time from the time when a subjective symptom appear in the patient, and examples thereof include, but not limited to, within 1 year, within 6 months, and within 3 months, for example.

As used herein, the terms, “protein,” “polypeptide,” “oligopeptide,” and “peptide”, are used interchangeably with the same meaning, and they refer to polymers of amino acids of any length. The polymer may be linear, branched or cyclic. The amino acids may be natural or non-natural, or may be modified amino acids. The term may also encompass those assembled into a complex of multiple polypeptide chains. The term also encompasses naturally or artificially modified amino acid polymers. Such modifications encompass, for example, disulfide bond formation, glycosylation, lipidation, acetylation, phosphorylation or any other manipulation or modification (e.g., conjugation with a labeling component). The subject definition also encompasses, for example, polypeptides including one or more analogs of amino acids (including, for example, unnatural amino acids), peptide-like compounds (e.g., peptoids) and other modifications known in the art. As used herein, an “amino acid” is a general term for organic compounds having an amino group and a carboxyl group. When the antibody according to the embodiment of the present invention includes a “specific amino acid sequence”, any amino acid in the amino acid sequence may be a chemically-modified amino acid. Furthermore, any amino acid in the amino acid sequence may form a salt or a solvate. Furthermore, any amino acid in the amino acid sequence may be of L-type or D-type. Even in such cases, the protein according to the embodiment of the present invention is considered to include the above-mentioned “specific amino acid sequence”. As for chemical modifications that amino acids included in proteins undergo in vivo, known are, for example, N-terminal modification (e.g., acetylation, myristoylation, etc.), C-terminal modification (e.g., amidation, glycosylphosphatidylinositol addition, etc.), side chain modifications (e.g., phosphorylation, glycosylation, etc.), or the like. It may be natural or non-natural as long as it satisfies the object of the present invention.

As used herein, a “chimera” (protein, rhodopsin) refers to a substance in a state in which genetic information derived from different organisms is mixed with each other in the same entity (in this case, protein, rhodopsin, etc.). The chimeric protein includes gene sequences derived from, for example, two or three or more organisms mixed therein. The sequence information contained in the chimeric protein may include a sequence other than the sequence derived from the organism to be mixed.

As used herein, the terms, “polynucleotide”, “oligonucleotide” and “nucleic acid”, are used interchangeably with the same meaning, and they refer to polymers of nucleotides of any length. The terms also include an “oligonucleotide derivative” or “polynucleotide derivative”. The “oligonucleotide derivative” or “polynucleotide derivative” refers to an oligonucleotide or polynucleotide containing a derivative of a nucleotide or having an unusual bond between nucleotides, and the terms are used interchangeably. Specific examples of such oligonucleotides include, for example, 2′-O-methyl-ribonucleotide, an oligonucleotide derivative in which a phosphate diester bond in an oligonucleotide is converted to a phosphorothioate bond, an oligonucleotide derivative in which a phosphate diester bond in an oligonucleotide is converted into an N3′-P5′phospholoamidate bond, an oligonucleotide derivative in which ribose and a phosphodiester bond in an oligonucleotide are converted into a peptide nucleic acid bond, an oligonucleotide derivative in which uracil in an oligonucleotide is substituted by C-5 propynyl uracil, an oligonucleotide derivative in which uracil in an oligonucleotide is substituted by C-5 thiazole uracil, an oligonucleotide derivative in which cytosine in an oligonucleotide is substituted by C-5 propynylcytosine, an oligonucleotide derivative in which cytosine in an oligonucleotide is substituted by phenoxazine-modified cytosine, an oligonucleotide derivative in which ribose in DNA is substituted by 2′-O-propyl ribose, and an oligonucleotide derivative in which ribose in an oligonucleotide is substituted by 2′-methoxyethoxyribose, and the like. Unless otherwise indicated, particular base sequences are also intended to include conservatively modified variants (e.g., degenerate codon substitutes) and complementary sequences thereof, similarly to the explicitly indicated sequences. Note that the sequences of nucleic acids are also referred to as nucleic acid sequences, nucleotide sequences, etc., in addition to base sequences, but they all have the same meaning. Specifically, the degenerate codon substitute may be achieved by creating a sequence in which the third position of one or more selected (or all) codons is substituted by a mixed base and/or deoxyinosine residue (Batzer et al., Nucleic Acid Res. 19: 5081(1991); Ohtsuka et al., J. Biol. Chem. 260: 2605-2608 (1985); Rossolini et al., Mol. Cell. Probes 8: 91-98 (1994)). In accordance with the context, the “nucleic acid” is also used herein interchangeably with genes, DNA such as cDNA, RNA such as mRNA, oligonucleotides, and polynucleotides. The “nucleotide” herein may be natural or non-natural. The nucleic acids can be DNA or RNA herein.

As used herein, a “gene” refers to an agent that defines a genetic trait, and the “gene” may refer to any of a “polynucleotide”, an “oligonucleotide” and a “nucleic acid”.

As used herein, “homology” of a gene refers to the degree of identity of two or more gene sequences to each other, and the concept of having “homology” generally refers to having a high degree of identity or similarity. The term, “identity”, refers to the equivalent degree of sequence of the same amino acid, while the term, “similarity”, refers to the equivalent degree of sequence, including amino acids of similar nature, in addition to the same amino acid. Thus, as the degree of the homology of two certain genes increases, the degree of the identity or similarity of their sequences increases. Whether or not two different genes have homology can be examined by direct sequence comparison or, in the case of nucleic acids, hybridization under stringent conditions. In a direct comparison between two gene sequences, those genes are homologous when the DNA sequences are typically at least 50% identical, preferably at least 70% identical, and more preferably at least 80%, 90%, 95%, 96%, 97%, 98% or 99% identical, between the gene sequences thereof. Thus, as used herein, a “homologue” or “homologous gene product” means a protein in another species, preferably a mammal, that exerts the same biological functions as the protein components of the complex further described herein. Such homologues are also sometimes referred to as “ortholog gene products”. It is understood that such homologues, homologous gene products, ortholog gene products and the like can also be used as long as these substances meet the object of the present invention.

Amino acids can be referred to herein by either their generally known three-letter symbols or the one-letter symbols recommended by the IUPAC-IUB Biochemical Nomenclature Commission. Nucleotides can also be referred to by the generally recognized one-letter codes. Herein, comparison of similarity, identity and homology of amino acid sequences and base sequences is calculated with default parameters using a tool for sequence analysis, BLAST. The identity search can be performed using, for example, NCBI's BLAST 2.2.28 (issued on Apr. 2, 2013) (Proc. Natl. Acad. Sci. USA 90: 5873-5877, 1993). The value of identity herein usually refers to the value obtained by performing alignment under the default conditions using the above BLAST. However, if a higher value is obtained by varying the parameters, the highest value obtained is set as the value for the identity. When identity is evaluated in multiple regions, the highest value among them is set as the value for the identity. Similarity refers to a numerical value that takes into account similar amino acids in addition to identity. Blastp can be used with default settings for the algorithm in the comparison between amino acid sequences in BLAST. The measurement results are quantified as Positives or Identities. The homology of the amino acid sequence and base sequence can be determined by the algorithm BLAST by Karlin and Altschul. Based on this algorithm, programs called BLASTN and BLASTX have been developed (Altschul et al. J. Mol. Biol. 215: 403-410, 1990). When the base sequence is analyzed by BLASTN based on BLAST, the parameters are set as, for example, score=100 and worldlength=12. When the amino acid sequence is analyzed by BLASTX based on BLAST, the parameters are set as, for example, score=50 and worldlength=3. When BLAST and Gapped BLAST programs are used, the default parameters of each program are used. Specific techniques of these analysis methods are known (http://www.ncbi.nlm.nih.gov).

The nucleic acid or protein as used herein may include a sequence in which one or more amino acids or nucleotides are substituted, deleted and/or added in the amino acid or base sequence of interest. In this regard, the term “one or more”, in SEQ ID NOs: 1 to 4 of the chimeric protein full-length amino acid sequence, typically means 50 amino acids or less, preferably 30 amino acids or less, and still more preferably 10 amino acids or less (e.g., 5 amino acids or less, 3 amino acids or less, or one amino acid). Further, “one or more”, in an amino acid sequence of a domain such as SEQ ID NOs: 5-7, typically means 6 amino acids or less, preferably 5 amino acids or less, and still more preferably 4 amino acids or less (e.g., 3 amino acids or less, 2 amino acids or less, and one amino acid). When maintaining the claimed biological activity of chimeric protein, it is desirable that an amino acid residue to be mutated be mutated to another amino acid which conserves the property of the amino acid side chain. Examples of properties of an amino acid side chain include hydrophobic amino acids (A, I, L, M, F, P, W, Y, V), hydrophilic amino acids (R, D, N, C, E, Q, G, H, K, S, T), amino acids with an aliphatic side chain (G, A, V, L, I, P), amino acids with a hydroxyl group containing side chain (S, T, Y), amino acids with a sulfur atom containing side chain (C, M), amino acids with a carboxylic acid and amide containing side chain (D, N, E, Q), amino acids with a base containing side chain (R, K, H), and amino acids with an aromatic containing side chain (H, F, Y, W) (each symbol within the parenthesis represents the one-letter code of an amino acid). These are also referred to herein as “conservative substitutions”. Note that a protein having an amino acid sequence modified by deletion, addition and/or substitution with another amino acid of one or more amino acid residues to the amino acid sequence, is known to maintain the biological activity thereof (Mark, D. F. et al., Proc. Natl. Acad. Sci. USA (1984) 81, 5662-5666; Zoller, M. J. & Smith, M. Nucleic Acids Research (1982) 10, 6487-6500; Wang, A. et al., Science 224, 1431-1433; Dalbadie-McFarland, G. et al., Proc. Natl. Acad. Sci. USA (1982) 79, 6409-6413). Therefore, in one embodiment of the present invention, “several” may be, for example, 10, 8, 6, 5, 4, 3, or 2, or may be less than or equal to any one of these numerical values. Chimeric protein with deletion etc. can be produced, for example, by a site-specific mutagenesis method, a random mutagenesis method, biopanning using an antibody phage library, or the like. As a site-specific mutagenesis method, KOD-Plus-Mutagenesis Kit (TOYOBO CO., LTD.), for example, can be used. It is possible to select an antibody having the same activity as the wild type, from the mutant-type antibody into which the deletion or the like has been introduced, by performing various characterizations, such as FACS analysis and ELISA.

In one embodiment of the present invention, the amino acid sequence and nucleic acid sequence of the chimeric protein of the present invention may have 70% or more, 80% or more, or 90% or more identity or similarity with the reference sequence. Regarding the amino acid sequence or base sequence herein, “70% or more” may be, for example, 70, 75, 80, 85, 90, 95, 96, 97, 98, 99% or more; “80% or more” may be, for example, 80, 85, 90, 95, 96, 97, 98, 99% or more; “90% or more” may be, for example, 90, 95, 96, 97, 98, 99% or more, or may be within the range of any two of the values. As for the “similarity”, the proportion of homologous amino acids between two or more amino acid sequences may be calculated according to methods known in the art. Before calculating the proportion, the amino acid sequences of the group of amino acid sequences to be compared are aligned, and gaps are introduced in some of the amino acid sequences if necessary to maximize the proportion of identical amino acids. Methods for alignment, methods for calculating proportions, comparison methods, and computer programs related thereto have been well known in the art (e.g., BLAST, GENETYX, etc.). The proportion of the same amino acids is calculated in the case of “identity”, whereas the proportion of similar amino acids is calculated in the case of “similarity”. Similar amino acids include, but are not limited to, amino acids that can be conservatively substituted.

As used herein, a “polynucleotide that hybridizes under stringent conditions” refers to well-known conditions commonly used in the art. Such a polynucleotide can be obtained by using a polynucleotide selected from the polynucleotides of the present invention as a probe and using a colony hybridization method, a plaque hybridization method, a Southern blot hybridization method, or the like. Specifically, the polynucleotide as above means such a polynucleotide that can be identified by performing hybridization at 65° C. in the presence of 0.7 to 1.0 M NaCl, using a filter with DNA immobilized from colonies or plaques, and then washing the filter under 65° C. conditions using a SSC (saline-sodium citrate) solution with a concentration of 0.1 to 2-fold (note that the composition of the 1-fold SSC solution is 150 mM sodium chloride and 15 mM sodium citrate). For the “stringent conditions”, the following conditions, for example, can be adopted: (1) use of low ionic strength and high temperature for washing (e.g., 0.015 M sodium chloride/0.0015 M sodium citrate/0.1% sodium dodecyl sulfate, at 50° C.); (2) use of denaturing agents, such as formamide, during hybridization (e.g., 50% (v/v) formamide and 0.1% bovine serum albumin/0.1% ficoll/0.1% polyvinylpyrrolidone/50 mM sodium phosphate buffer with pH of 6.5, and 750 mM sodium chloride, 75 mM sodium citrate, at 42° C.); or (3) incubation in a solution containing 20% formamide, 5×SSC, 50 mM sodium phosphate (pH 7.6), 5×Denhardt's solution, 10% dextran sulfate and 20 mg/ml denatured shear salmon sperm DNA at 37° C. overnight, followed by washing the filter with 1×SSC at about 37-50° C. Note that the formamide concentration may be 50% or higher. The washing time may be 5, 15, 30, 60 or 120 minutes, or more. Multiple factors such as temperature and salt concentration can be considered as factors that affect the stringency of the hybridization reaction, the details of which can be found in Ausubel et al., Current Protocols in Molecular Biology, Wiley Interscience Publishers, (1995). Examples of “highly stringent conditions” are 0.0015M sodium chloride, 0.0015M sodium citrate, at 65-68° C., or 0.015M sodium chloride, 0.0015M sodium citrate and 50% formamide at 42° C. As for hybridization, it can be carried out according to a method described in an experimental document, such as Molecular Cloning 2nd ed., Current Protocols in Molecular Biology, Supplement 1-38, DNA Cloning 1: Core Techniques, A Practical Approach, Second Edition, Oxford University Press (1995), or the like. Here, sequences containing only the A sequence or only the T sequence are preferably excluded from the sequences that hybridize under the stringent conditions. Moderately stringent conditions can be readily determined by one of ordinary skill in the art, based on, for example, the length of the DNA, as shown in Sambrook et al., Molecular Cloning: A Laboratory Manual, No. 3, Vol. 1, 7.42-7.45 Cold Spring Harbor Laboratory Press, 2001. Furthermore, with regard to nitrocellulose filters, included are use of hybridization conditions of 5×SSC, 0.5% SDS, 1.0 mM EDTA (pH 8.0) prewash solution, about 50% formamide at about 40-50° C., and 2×SSC-6×SSC (or other similar hybridization solution, such as Stark's solution, in about 50% formamide at about 42° C.), and washing conditions of about 60° C., 0.5×SSC and 0.1% SDS. Accordingly, the polypeptide used in the present invention also includes a polypeptide encoded by a nucleic acid molecule that hybridizes under highly or moderately stringent conditions to the nucleic acid molecule encoding the polypeptide specifically described in the present invention.

As used herein, a “purified” substance or biological factor (e.g., nucleic acid or protein) refers to one from which at least some of the factors naturally associated with the substance or biological factor have been removed. Therefore, the purity of the biological factor in the purified biological factor is usually higher (i.e., more enriched) than the purity of the biological factor in the state in which the biological factor is normally present. The term “purified” as used herein means that there are preferably at least 75% by weight, more preferably at least 85% by weight, even more preferably at least 95% by weight, and most preferably at least 98% by weight of biological factors of the same type. The substance or biological factor used in the present invention is preferably a “purified” substance. An “isolated” substance or biological factor (e.g., nucleic acid or protein) as used herein refers to one in which a factor naturally associated with the substance or biological factor has been substantially removed. The term “isolated” as used herein varies in accordance with its purpose and therefore does not necessarily have to be expressed in purity, but if necessary, the term means that there are preferably at least 75% by weight, more preferably at least 85% by weight, even more preferably at least 95% by weight, and most preferably at least 98% by weight of biological factors of the same type. The substance used in the present invention is preferably an “isolated” substance or biological factor.

As used herein, a “corresponding” amino acid or nucleic acid or moiety refers, in a polypeptide or polynucleotide molecule (e.g., rhodopsin), to an amino acid or nucleotide that has or is expected to have the same effect as a given amino acid or nucleotide or moiety in a polypeptide or polynucleotide that serves as a reference for comparison. In particular, as for an enzyme molecule, it refers to an amino acid that exists at a similar position in the active site and makes a similar contribution to catalytic activity, whereas as for a complex molecule, it refers to a corresponding moiety (e.g., heparan sulfate, etc.). In an antisense molecule, for example, it may be a similar moiety in the ortholog that corresponds to a particular moiety of the antisense molecule. The corresponding amino acid may be, for example, a specific amino acid that is cysteineized, glutathioneized, S—S bond formed, oxidized (e.g., methionine side chain oxidation), formylated, acetylated, phosphorylated, glycosylated, myristylated, and the like. Alternatively, the corresponding amino acid may be the amino acid responsible for dimerization. Such “corresponding” amino acids or nucleic acids may be regions or domains over a range. Thus, in such a case, they are referred to herein as a “corresponding” region or domain. Such a corresponding region or domain is useful when designing a complex molecule in the present invention.

As used herein, a “corresponding” gene (e.g., a polynucleotide sequence or molecule) refers, in a certain species, to a gene (e.g., a polynucleotide sequence or molecule) that has or is expected to have the same effect as a given gene in the species of reference for comparison. When there are multiple genes having such an action, those having the same evolutionary origin are referred to as the corresponding genes. Thus, the gene corresponding to a gene may be the ortholog of that gene. Thus, for each human rhodopsin, the corresponding rhodopsin can be found in other animals (particularly mammals). Such corresponding genes can be identified using techniques well known in the art. Thus, for example, with regard to a corresponding gene in a certain animal (e.g., a mouse), the gene of reference for the corresponding gene (e.g., rhodopsin, etc.) can be found by searching a database containing the sequences of the animal, with a sequence of SEQ ID NO: 1 to 17 or the like used as a query sequence.

As used herein, a “fragment” refers to a polypeptide or polynucleotide having a sequence length from 1 to n−1 with respect to a full-length polypeptide or polynucleotide (having the length of n). The length of the fragment can be appropriately varied in accordance with its purpose. For example, the lower limit of the length, in the case of a polypeptide, includes 3, 4, 5, 6, 7, 8, 9, 10, 15, 20, 25, 30, 40, 50 and more amino acids, and other lengths represented by integers not specifically listed here (e.g., 11) may also be appropriate as the lower limit. Furthermore, in the case of a polynucleotide, included are 5, 6, 7, 8, 9, 10, 15, 20, 25, 30, 40, 50, 75, 100 and more nucleotides, and other lengths represented by integers not specifically listed here (e.g., 11) may also be appropriate as the lower limit. It is understood herein that any fragment may fall within the scope of the present invention when the full length one, for example, functions as a marker or target molecule and the fragment itself also functions as a marker or target molecule.

According to the present invention, the term “activity” as used herein refers to the function of a molecule in the broadest sense. The activity generally includes, without intention of limitation, the biological, biochemical, physical or chemical function of the molecule. The activity includes, for example, enzyme activity, ability to interact with other molecules, ability to activate, promote, stabilize, inhibit, suppress or destabilize the function of other molecules, stability, and ability to localize to a specific intracellular location. Where applicable, the term also relates to the function of protein complexes in the broadest sense. As used herein, “biological activity” includes activation of photochemical reactions and the like.

As used herein, a “functional equivalent” refers to any entity having the same target function but a different structure with respect to the original entity of interest. It is thus understood that the functional equivalent of “rhodopsin” or a chimera thereof includes, not the rhodopsin or chimera thereof itself, but a mutant or variant (e.g., an amino acid sequence variant, etc.) of the rhodopsin or chimera thereof having the biological activity of the rhodopsin or chimera thereof, and further includes one that, at the time of action, can be transformed into rhodopsin or an antibody thereof or a mutant or variant of the rhodopsin or a chimera thereof (including, for example, a nucleic acid encoding rhodopsin or a chimera thereof or a mutant or variant of rhodopsin or a chimera thereof, and a vector, cell, etc., containing the nucleic acid). As the functional equivalent of the present invention, an amino acid sequence in which one or more amino acids are inserted, substituted and/or deleted, or added to one or both ends thereof can be used. As used herein, an “amino acid sequence in which one or more amino acids are inserted, substituted and/or deleted, or added to one or both ends thereof” means that it has been modified with substitution or the like of a plurality of amino acids that can occur naturally, by a well-known technical method such as site-specific mutagenesis, or by a natural mutation. The modified amino acid sequence can be, for example, one in which 1 to 30, preferably 1 to 20, more preferably 1 to 9, still more preferably 1 to 5, and particularly preferably 1 to 2 amino acids have been inserted, substituted or deleted, or added to one or both ends thereof. The modified amino acid sequence may preferably be such an amino acid sequence that has one or more (preferably one or several or 1, 2, 3, or 4) conservative substitutions in the rhodopsin amino acid sequence.

As used herein, an “agent”, “-agent” or “factor” (any of which corresponds to the word, agent, in English) may be used interchangeably in a broad sense, may be any substance or other element (e.g., energy, such as light, radioactivity, heat and electricity) that is capable of achieving the intended objective thereof. Examples of such substances include, without limitation, proteins, polypeptides, oligopeptides, peptides, polynucleotides, oligonucleotides, nucleotides, nucleic acids (including, for example, cDNA, DNA such as genomic DNA, RNA such as mRNA), polysaccharides, oligosaccharides, lipids, organic small molecules (e.g., hormones, ligands, messenger substances, organic small molecules, molecules synthesized by combinatorial chemistry, small molecules that can be used as pharmaceuticals (for example, small molecule ligands), etc.).

For oral administration, the agent may be formulated into various forms such as tablets, granules, fine granules, powder, and capsules for use. An additive commonly used in a formulation such as a binding agent, covering agent, excipient, lubricant, disintegrant, or humectant may also be included. In addition thereto, formulations for oral administration may be formulated as a liquid formulation such as an aqueous solution for internal use, suspension, emulsion, or syrup. The formulation may also be formulated as a dry formulation that is dissolved in a solvent upon use.

For parenteral administration, the agent may be formulated to be contained in a unit dose ampule or multidose container or tube. An additive such as a stabilizer, buffer, preservative, or isotonizing agent may also be included. A formulation for parenteral administration may also be formulated into a powder form that can be dissolved in a suitable carrier (sterilized water or the like) upon use.

Examples of parenteral administration include intravitreal administration, subconjunctival administration, intra-anterior chamber administration, and eye drops, and intravitreal administration is preferred. The composition and the like according to the present invention can be used for the treatment, prevention, suppression of progression, and the like by administration to humans using the aforementioned method.

As used herein, “treatment” refers to preventing the exacerbation of a disease or disorder (e.g., retinal degenerative disease) in the event of such a condition, preferably maintaining the status quo, more preferably alleviating, and even more preferably resolving, of the disease or disorder, including the possible exertion of a symptom improving or preventing effect on the patient's disease or one or more symptoms associated with the disease. Conducting diagnosis in advance and appropriate treatment is called “companion treatment”, and the diagnostic agent for that purpose is sometimes called “companion diagnostic agent”. Since the present invention targets genetic disorders, the gene may be tested in advance to treat the patient.

As used herein, a “therapeutic drug (agent)” refers, in a broad sense, to any agent capable of treating a target condition (for example, retinal degenerative disease). In one embodiment of the present invention, the “therapeutic drug” may be a pharmaceutical composition comprising an active ingredient and one or more pharmacologically acceptable carriers. The pharmaceutical composition can be manufactured, for example, by mixing an active ingredient with the above carrier and using any method known in the technical field of pharmaceutics. Further, the therapeutic drug is not limited in the form of use as long as it is used for treatment, and may be an active ingredient alone or a mixture of an active ingredient and any component. Further, the shape of the carrier is not particularly limited, and may be, for example, a solid or a liquid (e.g., a buffer solution).

As used herein, “prevention” refers, with regard to a disease or disorder (e.g., retinal degenerative disease), to preventing one from having such a condition before being in such a condition. The agent of the present invention can be used for diagnosis, and if necessary, the agent of the present invention can be used to prevent, for example, retinal degenerative diseases, or to take preventive measures. As used herein, a “preventive drug (drug)” refers, in a broad sense, to any drug that can prevent a target condition (for example, a disease such as retinal degenerative disease).

As used herein, a “kit” refers to a unit that is usually divided into two or more compartments and provides portions to be provided (e.g., test agents, diagnostic agents, therapeutic agents, antibodies, labels, instruction manuals, etc.). The form of the present kit is preferable when the purpose thereof is to provide a composition that should not be mixed and provided, but is preferably mixed and used immediately prior to use, for stability reasons or the like. It is advantageous for such a kit to comprise preferably an instruction, or a written explanation, describing how to use the portions to be provided (e.g., test agents, diagnostic agents, or therapeutic agents) or how the reagent should be processed. When the kit is used as a reagent kit in the present specification, the kit usually includes an instruction or the like describing how to use a test agent, a diagnostic agent, a therapeutic agent, an antibody, and the like.

As used herein, an “active ingredient” refers to an ingredient contained in an amount necessary for the composition of the present invention to attain a target effect, such as treatment, prevention or suppression of progress, and may also contain other ingredients as long as the effect is not compromised below the desired level. Further, the pharmaceuticals, compositions and the like of the present invention may be those that are formulated. In addition, the route of administration of the pharmaceuticals, compositions, etc. of the present invention may be oral or parenteral, and can be appropriately set according to the form of the formulation or the like.

As used herein, an “instruction” (including package inserts, labels used by the US FDA, etc.) refers to such an instruction that describes to a physician or other user how to use a method that uses the present invention. The instruction contains words instructing a detection method according to the present invention, how to use a diagnostic agent, or administration of pharmaceuticals or the like. In addition, the instruction may include words instructing oral administration or administration to the retina (for example, by injection) as the administration site. This instruction is prepared in accordance with the format prescribed by the regulatory agency of the country in which the present invention is implemented (for example, the Ministry of Health, Labor and Welfare in Japan, the Food and Drug Administration (FDA) in the United States, etc.), and the instruction clearly states that it has been approved by the regulatory agency. The instruction is a so-called package insert or label and is usually provided in a paper medium; however, without limitation thereto, the instruction may also be provided in a form of, for example, an electronic medium (e.g., a website provided on the Internet, and e-mail).

### Preferred Embodiments

Preferred embodiments of the present invention will be described below. It is understood that the embodiments provided below are provided for a better understanding of the present invention and the scope of the present invention should not be limited to the following description. Therefore, it is clear that those skilled in the art can appropriately make modifications within the scope of the present invention in consideration of the description in the present specification. It is also understood that the following embodiments of the present invention may be used alone or in combination.

(Chimeric Rhodopsin)

In one aspect, the present invention provides new uses for chimeric rhodopsin and also provides nucleic acid molecules. Any chimeric rhodopsin capable of achieving the objective of the present invention may be used as the chimeric rhodopsin of the present invention. The chimeric rhodopsin used in the present invention is typically a chimeric protein of an ion-transporting receptor rhodopsin and a G protein-coupled receptor rhodopsin. To explain a typical example, fusion of animal-derived G protein-coupled receptor rhodopsin with reusable microbial-derived ion-transporting receptor rhodopsin can acquire high activity via the endogenous G protein by the G protein-coupled receptor while retaining the function of repeated activation possessed by the microbial-derived ion-transporting receptor ion-channel receptor rhodopsin, which allows achieving of excellent treating, improving, preventing, and progression-suppressing effects on diseases, disorders and symptoms of the retina.

In one embodiment, as the ion-transporting receptor rhodopsin used in the chimeric protein of the present invention, an ion pumping receptor rhodopsin and an ion channeling receptor rhodopsin can be used. In a preferred embodiment, the ion-transporting receptor rhodopsin is preferably derived from microorganisms, and those from cyanobacteria (blue-green bacteria), for example, are typical ones. Examples thereof include rhodopsin derived from microorganisms belonging to eubacteria, such as the genus Gloeobacter, and eukaryotes, such as the genus Volvox, genus Chlamydomonas, and genus Guillardia. Examples of the genus Gloeobacter include Gloeobacter violaceus and the like. Examples of the genus Volvox include Volvox carteri and the like. Examples of the genus Chlamydomonas include Chlamydomonas reinhardtii and the like. Examples of the genus Guillardia include Guillardia theta and the like.

In one embodiment, the G protein-coupled receptor rhodopsin used in the chimeric protein of the present invention is typically derived from animals, and rhodopsin derived from rodents, artiodactyls, cloven-hoofed animals, primates, carnivores, and the like is preferable, rhodopsin derived from artiodactyls or primates is more preferable, and rhodopsin derived from primates is still more preferable. In addition, preferable G protein-coupled receptor rhodopsin includes, for example, rhodopsin derived from bovine, human, mouse, rat, cat, dog, pig, sheep, horse and the like. Of these, bovine or human-derived rhodopsin is particularly preferable.

In a certain embodiment, the chimeric protein of the present invention is a chimeric protein of an ion-transporting receptor rhodopsin and a G protein-coupled receptor rhodopsin, and has a seven-time-transmembrane structure. In the present invention, the chimeric protein of the ion-transporting receptor rhodopsin and the G protein-coupled receptor rhodopsin is preferably designed to highly exert both: a function of repeatedly activating the ion-transporting receptor rhodopsin; and the G protein activity by the G protein-coupled receptor rhodopsin. From this point of view, the chimeric protein of the present invention maintains high activity of both, and particularly exhibits high visual function restoration ability, and thus, the chimeric protein of the present invention is preferably a chimeric protein in which the amino acid sequences of the second loop on the cytoplasmic side and/or the third loop on the cytoplasmic side of the amino acid sequences of the ion-transporting receptor rhodopsin are substituted by the amino acid sequences of the second loop on the cytoplasmic side and/or the third loop on the cytoplasmic side of the G protein-coupled receptor rhodopsin. Note that the “second loop on the cytoplasmic side” and the “third loop on the cytoplasmic side” refer to loops located second from the N-terminal side and third from the N-terminal side of the seven loops, respectively.

In one embodiment, it is advantageous for the chimeric protein of the present invention to have an amino acid sequence in which glutamic acid corresponding to position 132 of the amino acid sequence of SEQ ID NO: 8 (GR) is substituted by glutamine. Examples of glutamine-substituted amino acid sequences include, but are not limited to, the amino acid sequences encoded by SEQ ID NOs: 1 and 2 and SEQ ID NO: 10.

The method for obtaining a nucleic acid, such as DNA, of the present invention is not particularly limited, and examples thereof include a method of obtaining cDNA by reverse transcription from mRNA (for example, RT-PCR method), a method of preparation from genomic DNA, a method of synthesis by chemical synthesis, a method of isolation from a genomic DNA library or a cDNA library, and other known methods (see, for example, Japanese Laid-Open Publication No. Hei 11-29599).

Herein, the chimeric protein can be prepared, for example, by using a transformant into which an expression vector comprising a nucleic acid, such as DNA, encoding the above-mentioned chimeric protein has been introduced. For example, first, this transformant is cultured under appropriate conditions to synthesize a chimeric protein encoded by the nucleic acid, such as DNA. Then, the synthesized protein is recovered from the transformant or the culture medium, thereby acquiring the chimeric protein of the present invention.

More specifically, the chimeric protein can be prepared by inserting a DNA encoding the chimeric protein as described above into an appropriate expression vector. An “appropriate expression vector” may be any vector that can replicate, retain or self-proliferate in various hosts of prokaryotes and/or eukaryotes, and can be appropriately selected in accordance with the purpose of use. For example, a high copy vector can be selected when a large amount of nucleic acid such as DNA is to be obtained, while an expression vector can be selected when a polypeptide (chimeric protein) is to be obtained. Specific examples thereof include, without particular limitation, known vectors described in Japanese Laid-Open Publication No. Hei 11-29599.

In addition, the expression vector can be used, not only for the synthesizing of chimeric proteins, but also for the composition of the present invention or the like. Specifically, the composition of the present invention or the like may contain an expression vector in which a nucleic acid encoding the amino acid sequence of the above-mentioned chimeric protein is incorporated as an active ingredient. The direct introduction of such an expression vector into humans can be used for the treatment, prevention and suppression of progression of diseases, disorders or symptoms of the retina. As the vector in this case, a vector that can be introduced into human cells is used. As such a vector, preferable are, for example, an adeno-associated virus vector (AAV vector) and a lentiviral vector.

The method for introducing the vector can be appropriately selected in accordance with the type of vector and host, and the like. Specific examples thereof include, but are not limited to, known methods such as a protoplast method and a competent method when a bacterium is used as a host (see, for example, Japanese Laid-Open Publication No. Hei 11-29599). When the expression vector is used as an active ingredient of the visual function regenerating agent or the visual function deterioration preventing agent of the present invention, the introduction can be achieved by injecting the above AAV vector or the like into the eye, for example.

The hosts into which the expression vector is introduced may be any hosts that are compatible with the expression vector and can be transformed. Specific examples thereof include, but are not particularly limited to, bacteria, yeast, animal cells, insect cells, and other known natural cells or artificially established cells (see Japanese Laid-Open Publication No. Hei 11-29599), or humans, mice and other animals. The culturing of transformants can be performed by appropriately selecting a medium form from known nutrient media, and by appropriately adjusting the temperature, pH of the nutrient medium, culture time and the like, in accordance with the type of transformant, and the like (see, for example, Japanese Laid-Open Publication No. Hei 11-29599).

The methods for isolating and purifying the chimeric protein are not particularly limited, and examples of such methods include known methods such as methods that utilize solubility, methods that utilize a difference in molecular weights, and methods that utilize electric charges (see, for example, Japanese Laid-Open Publication No. Hei 11-29599).

In a specific embodiment, the chimeric protein of the present invention has any of the following amino acid sequences:

(a) an amino acid sequence set forth in SEQ ID NOs: 1-4 or a fragment thereof;
(b) an amino acid sequence having at least 80% identity to (a); and
(c) an amino acid sequence with one or more amino acids substituted, added and/or deleted with respect to (a) or (b), and also has biological activity. Alternatively, the chimeric protein of the present invention preferably has an amino acid sequence encoded by any of the following nucleic acids:
(aa) a nucleic acid having a base sequence encoding an amino acid sequence set forth in any of SEQ ID NOs: 1-4 or a base sequence set forth in SEQ ID NO: 10;
(bb) a nucleic acid having a base sequence that can hybridize under stringent conditions with a base sequence complementary to a base sequence encoding an amino acid sequence set forth in any of SEQ ID NOs: 1-4 or a base sequence set forth in SEQ ID NO: 10;
(cc) a nucleic acid having a base sequence encoding an amino acid sequence in which one or more amino acids are substituted, deleted and/or added in the amino acid sequence set forth in any of SEQ ID NOs: 1-4, and having biological activity; and
(dd) a nucleic acid consisting of a base sequence encoding an amino acid sequence having 90% or more homology with an amino acid sequence set forth in any of SEQ ID NOs: 1-4 and having biological activity, or any of the following:
(aaa) a base sequence set forth in SEQ ID NO: 10 or a fragment thereof;
(bbb) a nucleic acid having at least 80% identity to (aaa);
(ccc) a base sequence with one or more nucleotides substituted, added and/or deleted with respect to (aaa) or (bbb); and
(ddd) a base sequence that hybridizes to any of (aaa) to (ccc) under stringent conditions, and the chimeric protein also has biological activity.

In one embodiment, the nucleic acid encoding the chimeric protein of the present invention is preferably any of the following:

(aaa) a base sequence set forth in SEQ ID NO: 10 or a fragment thereof;
(bbb) a nucleic acid having at least 80% identity to (aaa);
(ccc) a base sequence in which one or more nucleotides are substituted, added and/or deleted with respect to (aaa) or (bbb); and
(ddd) a base sequence that hybridizes to any of (aaa) to (ccc) under stringent conditions, and the chimeric protein also has biological activity.

Alternatively, the second loop on the cytoplasmic side of the G protein-coupled receptor rhodopsin described above is preferably a loop having an amino acid sequence encoded by any of the nucleic acids described below:

(i) a nucleic acid having a base sequence encoding an amino acid sequence set forth in SEQ ID NO: 5 or 6;
(ii) a nucleic acid having a base sequence that can hybridize under stringent conditions with a base sequence complementary to a base sequence encoding an amino acid sequence set forth in SEQ ID NO: 5 or 6;
(iii) a nucleic acid having a base sequence encoding an amino acid sequence in which one or more amino acids are substituted, deleted and/or added in an amino acid sequence set forth in SEQ ID NO: 5 or 6; and
(iv) a nucleic acid consisting of a base sequence encoding an amino acid sequence having 90% or more homology with an amino acid sequence set forth in SEQ ID NO: 5 or 6, or the nucleic acid encoding the second loop on the cytoplasmic side of the G protein-coupled receptor rhodopsin is preferably any of the following:
(i) a nucleic acid having a base sequence encoding an amino acid sequence set forth in SEQ ID NO: 5 or 6;
(ii) a nucleic acid having a base sequence that can hybridize under stringent conditions with a base sequence complementary to a base sequence encoding an amino acid sequence set forth in SEQ ID NO: 5 or 6;
(iii) a nucleic acid having a base sequence encoding an amino acid sequence in which one or more amino acids are substituted, deleted and/or added in an amino acid sequence set forth in SEQ ID NO: 5 or 6;
(iv) a nucleic acid consisting of a base sequence encoding an amino acid sequence having 90% or more homology with an amino acid sequence set forth in SEQ ID NO: 5 or 6;
(x) a nucleic acid having a base sequence set forth in SEQ ID NO: 11 or SEQ ID NO: 12 or a fragment thereof;
(y) a nucleic acid having at least 80% identity to (x);
(z) a nucleic acid with one or more nucleotides substituted, added and/or deleted with respect to (x) or (y); and
(w) a nucleic acid that hybridizes to any of (x) to (z) under stringent conditions, and

the loop also has biological activity.

The third loop on the cytoplasmic side of the G protein-coupled receptor rhodopsin described above is preferably a loop having an amino acid sequence encoded by any of the nucleic acids described below:

(l) a nucleic acid having a base sequence encoding the amino acid sequence set forth in SEQ ID NO: 7;
(k) a nucleic acid having a base sequence that can hybridize under stringent conditions with a base sequence complementary to a base sequence encoding the amino acid sequence set forth in SEQ ID NO: 7;
(m) a nucleic acid having a base sequence encoding an amino acid sequence in which one or more amino acids are substituted, deleted and/or added in the amino acid sequence set forth in SEQ ID NO: 7; and
(n) a nucleic acid consisting of a base sequence encoding an amino acid sequence having 90% or more homology with the amino acid sequence set forth in SEQ ID NO: 7.

Alternatively, the nucleic acid encoding the third loop on the cytoplasmic side of the G protein-coupled receptor rhodopsin is preferably any of the following:

(l) a nucleic acid having a base sequence encoding the amino acid sequence set forth in SEQ ID NO: 7;
(k) a nucleic acid having a base sequence that can hybridize under stringent conditions with a base sequence complementary to a base sequence encoding the amino acid sequence set forth in SEQ ID NO: 7;
(m) a nucleic acid having a base sequence encoding an amino acid sequence in which one or more amino acids are substituted, deleted and/or added in the amino acid sequence set forth in SEQ ID NO: 7;
(n) a nucleic acid consisting of a base sequence encoding an amino acid sequence having 90% or more homology with the amino acid sequence set forth in SEQ ID NO: 7;
(xx) a nucleic acid having a base sequence set forth in SEQ ID NO: 13 or a fragment thereof;
(yy) a nucleic acid having at least 80% identity to (xx);
(zz) a nucleic acid with one or more nucleotides substituted, added and/or deleted with respect to (xx) or (yy); or
(ww) a nucleic acid that hybridizes to any of (xx) to (zz) under stringent conditions, and

the loop also has biological activity.

The present invention also provides a nucleic acid having one of the following:

(a) a nucleic acid having a base sequence set forth in SEQ ID NO: 10 or a fragment thereof;
(b) a nucleic acid having at least 80% identity to (a);
(c) a nucleic acid with one or more nucleotides substituted, added and/or deleted with respect to (a) or (b); and
(d) a nucleic acid that hybridizes to any of (a) to (c) under stringent conditions, where

the protein encoded by the nucleic acid has biological activity.

As used herein, typical examples of “biological activity” can include the function of the G protein-coupled receptor (e.g., membrane transfer efficiency) that the loop thereof has, and in addition, the prevention and suppression of progression of retinal diseases (e.g., retinitis pigmentosa), the visual cognitive behavioral functions (e.g., improvement in light-dark determination functions, improvement in bright spot evading functions, and/or crisis avoidance functions), and the function capable of exerting effects for augmenting visual acuity. The biological activity in the case of loops can include, but are not limited to, functions such as conformational compatibility and membrane transfer efficiency. Alternatively, the functions of the loop may be evaluated by the functions of the incorporated protein as a whole (herein, rhodopsin).

In the present invention, the chimeric protein of the present invention and the nucleic acid encoding the same have been found to be used for the purpose of preventing or suppressing the progression of diseases, disorders or symptoms of the retina, for the purpose of improving visual cognitive behavioral functions (e.g., improvement in light-dark determination functions, improvement in bright spot evading functions, and/or crisis avoidance functions), and for the purpose of providing visual function augmenting effects, such as improving the visual acuity.

While one of the eye diseases for which there is no cure to date is retinitis pigmentosa, atrophic age-related macular degeneration, and other retinal degenerative diseases, radical cures for these diseases may be provided by the present invention. Globally, the total number of patients with these diseases is said to exceed 130 million, while retinitis pigmentosa is the third leading cause, and age-related macular degeneration is the fourth leading cause, of acquired blindness in Japan. The development of a therapeutic method has been long desired due to the large number of such patients and the severity of visual impairment, which may be solved by the present invention.

Like the central nervous system, the photoreceptor cells, which are the primary neurons of vision, cannot be regenerated once they are lost. In retinitis pigmentosa and atrophic age-related macular degeneration, however, bipolar cells and retinal ganglion cells, which are the secondary and tertiary neurons of vision, are retained, which is considered to be one of the factors for the effectiveness of the present invention. The present invention is a gene transfer therapy using optogenetics, which can be expected to have a safe and long-term visual sense restoration effect with little invasiveness. Highly efficient and safe visual sense restoration has become possible by using the original, more physiological phototransmission pathways that utilize the endogenous G protein signal cascade and channels, which is completely different from the conventional method of introducing photoactivated ion channels. The conventional method of introducing photoactivated ion channels has been restoration for patients with already advanced retinal degeneration, whereas the present method does not require the metabolic restoration system of retinal called Visual Cycle, which is necessary for normal light transmission. Accordingly, the present method can also be expected to have an effect of suppressing the progression of retinal degeneration. This has proved that the present invention can be applied, not only to patients with advanced retinal degeneration, but also to the prevention of progression in patients in the early stage.

### (Prevention, or Suppression of Progression, of Retinal Disease, Disorder or Symptom)

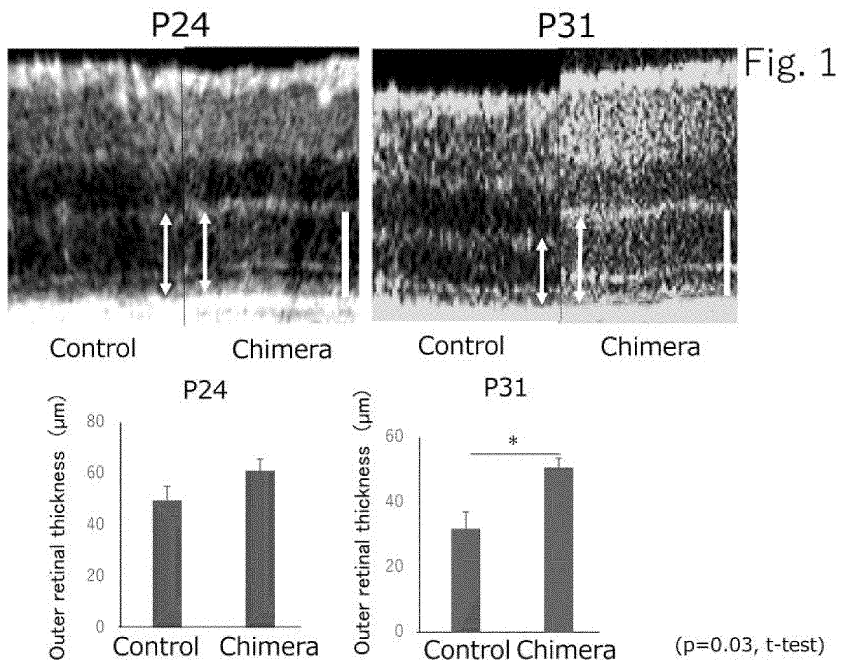

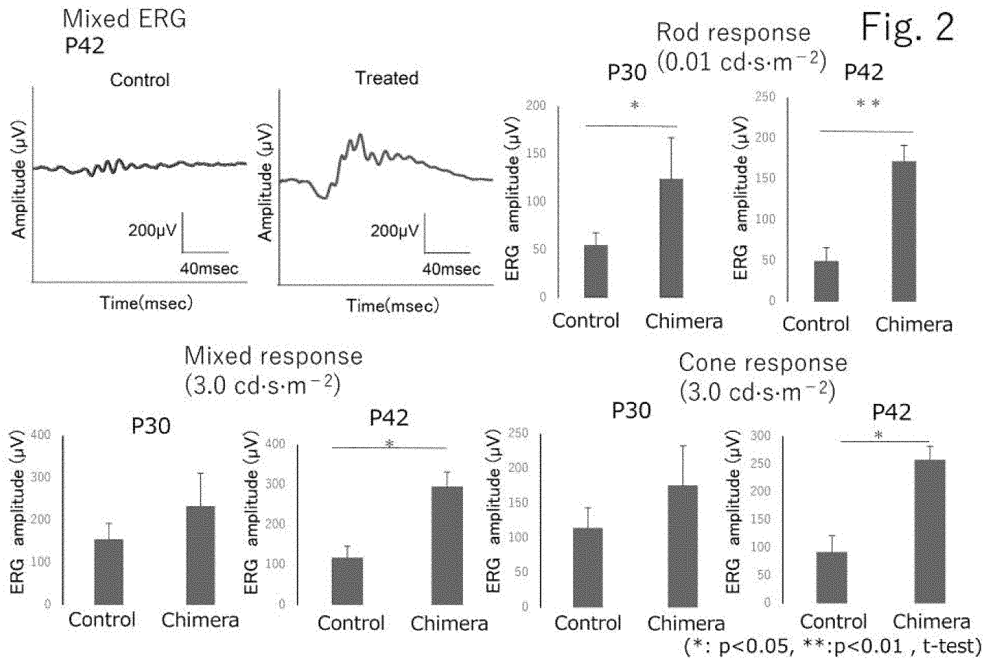

In one aspect, the present invention provides a composition for preventing, or suppressing the progression of, a disease, disorder or symptom of the retina, comprising a nucleic acid encoding a chimeric protein of an ion-transporting receptor rhodopsin and a G protein-coupled receptor rhodopsin. As the chimeric protein used in this aspect of the present invention, those of any embodiment described in the section (Chimeric Rhodopsin) can be utilized. The prevention or suppression of progression of diseases, disorders or symptoms of the retina, represented by the suppression of the progression of retinitis pigmentosa, in the present invention, has been confirmed by the demonstration in the photoreceptor thinning experiments shown in Example 1 and FIGS. 1 and 2.

In another aspect, the present invention provides a composition for preventing or suppressing the progression of a disease, disorder or symptom of the retina, comprising a chimeric protein of an ion-transporting receptor rhodopsin and a G protein-coupled receptor rhodopsin. As the chimeric protein used in this aspect of the present invention, those of any embodiment described in the section (Chimeric Rhodopsin) can be utilized.

In yet another aspect, provided is: a nucleic acid encoding a chimeric protein of an ion-transporting receptor rhodopsin and a G protein-coupled receptor rhodopsin, for preventing or suppressing the progression of a disease, disorder or symptom of the retina; or a chimeric protein of an ion-transporting receptor rhodopsin and a G protein-coupled receptor rhodopsin, for preventing or suppressing the progression of a disease, disorder or symptom of the retina. As the chimeric protein used in this aspect of the present invention, those of any embodiment described in the section (Chimeric Rhodopsin) can be utilized.

In yet another aspect, the present invention provides: a method for preventing or suppressing the progression of a disease, disorder or symptom of the retina in a subject, the method comprising the step of administering an effective amount of a nucleic acid encoding a chimeric protein of an ion-transporting receptor rhodopsin and a G protein-coupled receptor rhodopsin to the subject; or a method for preventing or suppressing the progression of a disease, disorder or symptom of the retina in a subject, the method comprising the step of administering an effective amount of a chimeric protein of an ion-transporting receptor rhodopsin and a G protein-coupled receptor rhodopsin to the subject. As the chimeric protein used in this aspect of the present invention, those of any embodiment described in the section (Chimeric Rhodopsin) can be utilized.

In yet another aspect, the present invention provides: use of a nucleic acid encoding a chimeric protein of an ion-transporting receptor rhodopsin and a G protein-coupled receptor rhodopsin, in the manufacture of pharmaceuticals for preventing or suppressing the progression of a disease, disorder or symptom of the retina; or use of a chimeric protein of an ion-transporting receptor rhodopsin and a G protein-coupled receptor rhodopsin, in the manufacture of pharmaceuticals for preventing or suppressing the progression of a disease, disorder or symptom of the retina.

In one embodiment, said disease, disorder or symptom is retinal degenerative disease. As the retinal degenerative disease, for example, retinitis pigmentosa and age-related macular degeneration are preferably advantageous, and retinitis pigmentosa is more preferably advantageous.

In a preferred embodiment, the retinitis pigmentosa targeted by the present invention is autosomal dominantly inherited and is preferably RHO autosomal preferentially inherited.

In a preferred embodiment, the present invention is used for the purpose of preventing or suppressing the progression of retinitis pigmentosa.

In a preferred embodiment, the present invention is preferably, but not limited to, administered to a subject before or immediately after the onset of a disease, disorder or symptom, such as, within 1 year, preferably within 6 months, within 3 months or within 1 month, from the onset (e.g., when subjective symptoms appear), for example.

In one particular embodiment, the composition or vector of the invention is administered once. It has been confirmed that the present invention is effective when administered once, where the compliance with patients is considered to be favorable.

In one particular embodiment, the amount of the vector used in the present invention is 0.1×10¹¹ to 10×10¹¹ vg/eye unit dose, where the lower limit thereof may be, for example, 0.01×10¹¹ vg/eye, 0.02×10¹¹ vg/eye, 0.03×10¹¹ vg/eye, 0.04×10¹¹ vg/eye, 0.05×10¹¹ vg/eye, 0.06×10¹¹ vg/eye, 0.07×10¹¹ vg/eye, 0.08×10¹¹ vg/eye, 0.09×10¹¹ vg/eye, 0.1×10¹¹ vg/eye, 0.2×10¹¹ vg/eye, 0.3×10¹¹ vg/eye, 0.4×10¹¹ vg/eye, 0.5×10¹¹ vg/eye or the like, while the upper limit thereof may be, for example, 2×10¹¹ vg/eye, 3×10¹¹ vg/eye, 4×10¹¹ vg/eye, 5×10¹¹ vg/eye, 6×10¹¹ vg/eye, 7×10¹¹ vg/eye, 8×10¹¹ vg/eye, 9×10¹¹ vg/eye, 10×10¹¹ vg/eye, 15×10¹¹ vg/eye, 20×10¹¹ vg/eye, 30×10¹¹ vg/eye, 40×10¹¹ vg/eye, 50×10¹¹ vg/eye or the like.

### (Improvement of Visual Cognitive Behavioral Function)

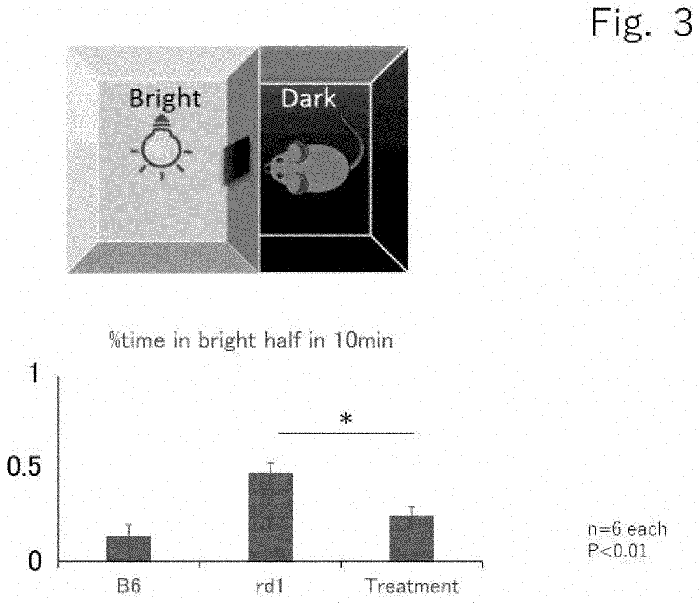

In one aspect, the present invention provides a composition for improving a visual cognitive behavioral function (e.g., improvement in a light-dark determination function, improvement in a bright spot evading function, and/or a crisis avoidance function), comprising a nucleic acid encoding a chimeric protein of an ion-transporting receptor rhodopsin and a G protein-coupled receptor rhodopsin. As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect. Functions such as improving visual cognitive behavioral functions (e.g., improvement in light-dark determination functions, improvement in bright spot evading functions, and/or crisis avoidance functions) have been verified with experimental models in the present invention, where the present invention is considered to exert significant effects. The effects for the visual cognitive behavioral functions (e.g., improvement in light-dark determination functions, improvement in bright spot evading functions, and/or crisis avoidance functions) have been demonstrated as a result of the testing by the light-dark transition test (LDT) demonstrated in Example 2 (see FIG. 3). The visual cognitive behavioral functions are such functions that can be confirmed by, not only confirming the photosensitivity of visual organs, but also verifying whether the functions actually appear as actions in animal models, etc. One of the achievements of the present invention is considered to be the verification achieved by the experiment as in Example 2 (see FIG. 3). The improvement in the visual cognitive behavioral functions includes improvement, enhancement, augmentation or the like of visual acuity, contrast sensitivity, light-dark adaptation, color vision, etc.

In another aspect, the present invention provides a composition for improving a visual cognitive behavioral function (e.g., improvement in a light-dark determination function, improvement in a bright spot evading function, and/or a crisis avoidance function), comprising a chimeric protein of an ion-transporting receptor rhodopsin and a G protein-coupled receptor rhodopsin. As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

In another aspect, the present invention provides a nucleic acid encoding a chimeric protein of an ion-transporting receptor rhodopsin and a G protein-coupled receptor rhodopsin, for improving a visual cognitive behavioral function (e.g., improvement in a light-dark determination function, improvement in a bright spot evading function, and/or a crisis avoidance function). As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

In yet another aspect, the present invention provides a chimeric protein of an ion-transporting receptor rhodopsin and a G protein-coupled receptor rhodopsin, for improving a visual cognitive behavioral function (e.g., improvement in a light-dark determination function, improvement in a bright spot evading function, and/or a crisis avoidance function). As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

In another aspect, the present invention provides a method for improving a visual cognitive behavioral function (e.g., improvement in a light-dark determination function, improvement in a bright spot evading function, and/or a crisis avoidance function) in a subject, the method comprising the step of administering an effective amount of a nucleic acid encoding a chimeric protein of an ion-transporting receptor rhodopsin and a G protein-coupled receptor rhodopsin to the subject. As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

In yet another aspect, the present invention provides a method for improving a visual cognitive behavioral function (e.g., improvement in a light-dark determination function, improvement in a bright spot evading function, and/or a crisis avoidance function) in a subject, the method comprising the step of administering an effective amount of a chimeric protein of an ion-transporting receptor rhodopsin and a G protein-coupled receptor rhodopsin to the subject. As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

In yet another aspect, the present invention provides use of a nucleic acid encoding a chimeric protein of an ion-transporting receptor rhodopsin and a G protein-coupled receptor rhodopsin, in the manufacture of pharmaceuticals for improving a visual cognitive behavioral function (e.g., improvement in a light-dark determination function, improvement in a bright spot evading function, and/or a crisis avoidance function). As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

In yet another aspect, the present invention provides use of a chimeric protein of an ion-transporting receptor rhodopsin and a G protein-coupled receptor rhodopsin, in the manufacture of pharmaceuticals for improving a visual cognitive behavioral function (e.g., improvement in a light-dark determination function, improvement in a bright spot evading function, and/or a crisis avoidance function). As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

### (Visual Function Enhancement and Visual Acuity Improvement)

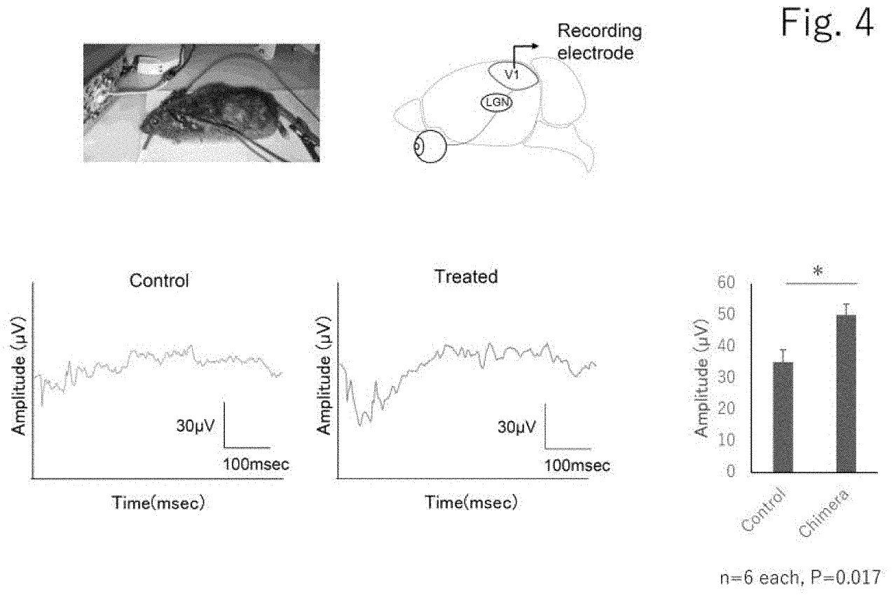

In one aspect, the present invention provides a composition for improving visual acuity, comprising a nucleic acid encoding a chimeric protein of an ion-transporting receptor rhodopsin and a G protein-coupled receptor rhodopsin. As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect. The function of improving visual acuity has been verified with experimental models in the present invention, where the present invention is considered to exert significant effects. The enhancement of visual functions, such as improvement in visual acuity, has been confirmed by the demonstration in the experiments of the visual evoked potential VEP represented by Example 3 and FIG. 4.

In another aspect, the present invention provides a composition for enhancing a visual function (e.g., improving visual acuity), comprising a chimeric protein of an ion-transporting receptor rhodopsin and a G protein-coupled receptor rhodopsin. As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

In yet another aspect, the present invention provides a nucleic acid encoding a chimeric protein of an ion-transporting receptor rhodopsin and a G protein-coupled receptor rhodopsin, for enhancing a visual function (e.g., improving visual acuity). As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

In yet another aspect, the present invention provides a chimeric protein of an ion-transporting receptor rhodopsin and a G protein-coupled receptor rhodopsin, for enhancing a visual function (e.g., improving visual acuity). As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

In yet another aspect, the present invention provides a method for enhancing a visual function (e.g., improving visual acuity) in a subject, the method comprising the step of administering an effective amount of a nucleic acid encoding a chimeric protein of an ion-transporting receptor rhodopsin and a G protein-coupled receptor rhodopsin to the subject. As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

In yet another aspect, the present invention provides a method for enhancing a visual function (e.g., improving visual acuity) in a subject, the method comprising the step of administering an effective amount of a chimeric protein of an ion-transporting receptor rhodopsin and a G protein-coupled receptor rhodopsin to the subject. As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

In yet another aspect, the present invention provides use of a nucleic acid encoding a chimeric protein of an ion-transporting receptor rhodopsin and a G protein-coupled receptor rhodopsin, in the manufacture of pharmaceuticals for enhancing a visual function (e.g., improving visual acuity). As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

In yet another aspect, the present invention provides use of a chimeric protein of an ion-transporting receptor rhodopsin and a G protein-coupled receptor rhodopsin, in the manufacture of pharmaceuticals for enhancing a visual function (e.g., improving visual acuity). As the chimeric protein used in this aspect, those of any embodiment described in the section (Chimeric Rhodopsin) can be utilized. Furthermore, it is understood that therapeutic forms of any embodiment described in the section (Prevention or Suppression of Progression of Retinal Disease, Disorder or Symptom) can be applied as the therapeutic form used in this aspect.

As used herein, the term, “or”, is used when “at least one or more” of the matters listed in the sentences can be employed. When explicitly described herein as “within the range of two of the values”, the range also includes the two values themselves.

Reference literatures such as scientific literatures, patents, and patent applications cited herein are incorporated herein by reference to the same extent that the entirety of each document is specifically described.

As described above, the present invention has been explained while showing preferred embodiments to facilitate understanding. The present invention is explained hereinafter based on Examples. The above explanation and the following Examples are not provided to limit the present invention, but for the sole purpose of exemplification. Thus, the scope of the present invention is not limited to the embodiments or the Examples specifically described herein and is limited only by the scope of claims.

### Examples

Examples will be described hereinafter. The handling of animals used in the following examples was carried out, if necessary, based on the Declaration of Helsinki, in compliance with the standards and other relevant ethical standards and guidelines as stipulated by Keio University and others. As for reagents, while those specifically described in Examples were used, these reagents can be substituted by equivalent products of other manufacturers (such as, Sigma-Aldrich, Wako Pure Chemical, Nacalai, R & D Systems and USCN Life Science Inc.).

### (Vector Preparation)

The DNA encoding the chimeric protein (GR/BvRh) was produced as follows.

The sequence corresponding to the 137th to 145th amino acids from the N-terminal, which corresponds to the second loop on the cytoplasmic side of Gloeobacter violaceus Rhodopsin (GR) (SEQ ID NO: 8), was substituted by the sequence corresponding to the 137th to 145th amino acids of bovine rhodopsin (BvRh) (SEQ ID NO: 9), and the sequence corresponding to 198th to 206th amino acids from the N-terminal, which corresponds to the third loop on the cytoplasmic side of GR, was substituted by the sequence corresponding to the 225th to 252nd amino acids of the bovine rhodopsin. Furthermore, DNA encoding a chimeric protein, in which glutamic acid, or the 132nd amino acid of GR, was substituted by glutamine, was inserted into the pCDNA3.1 vector. Alternatively, nucleic acids having the base sequence set forth in SEQ ID NO: 10 were generated and inserted, as the DNA encoding the chimeric protein, into the pCDNA3.1 vector HindIII/XbaI site. The base sequence set forth in SEQ ID NO: 10 was generated as follows: the sequence corresponding to the 137th to 145th amino acids from the N-terminal, which corresponds to the second loop on the cytoplasmic side of Gloeobacter violaceus Rhodopsin (GR) (SEQ ID NO: 8), was substituted by the base sequence set forth in SEQ ID NO: 12 corresponding to the second loop of bovine rhodopsin (BvRh) (SEQ ID NO: 9) (the encoding of the amino acid sequence set forth in SEQ ID NO: 6), and the sequence corresponding to 198th to 206th amino acids from the N-terminal, which corresponds to the third loop on the cytoplasmic side of GR, was substituted by the base sequence set forth in SEQ ID NO: 13 corresponding to the third loop of the bovine rhodopsin (the encoding of the amino acid sequence set forth in SEQ ID NO: 7), thereby producing the base sequence. The production of the mutant was conducted using the quick change method. Note that the sequence portion adopted for bovine rhodopsin completely matches the amino acid sequence of human rhodopsin, and thus, the sequence portion may be referred to as human rhodopsin without any problem.

The EGFP or GR/BvRh gene was subcloned into the AAV2 shuttle plasmid, and AAV2-CAGGS-EGFP-WPRE-pA (vector for the expression of EGFP) and AAV2-CAGGS-GR/BvRh-WPRE-pA (vector for the expression of chimeric protein) were produced as virus expression constructs. Viral vector packaging was performed by transfecting HEK293 cells with three types of plasmids, vector plasmid, AAV vector plasmid and adenovirus helper plasmid; and the cesium chloride method was used to purify the viral vector. Note that, with regard to the vector, the “ITR” is an abbreviation for “Inverted Terminal Repeat”. The “CAGGS” is a sequence of regions of the CAG promoter. The “WPRE” is an abbreviation for “woodchuck hepatitis virus post-transcriptional regulatory element”. The “pA” means a peptide tag. The “EGFP” is an abbreviation for “enhanced green fluorescent protein”.

Note that all the numerical values in the examples show an average±SEM.

Hereinafter, using an adeno-associated virus vector (AAV) (type 2 or DJ) with a chimeric rhodopsin gene incorporated therein, intravitreal injection or subretinal injection was performed under anesthesia for retinitis pigmentosa model mice (rd1 mouse or P23H mouse), followed by inducing the expression of chimeric rhodopsin in retinal ganglion cells or photoreceptor cells, thereby conducting the confirmation of the visual sense restoration and prevention effects using electroretinogram (ERG) and optical coherence tomography (OCT).

### Example 1: Photoreceptor Thinning Experiments

In the present example, experiments for thinning photoreceptor cells were conducted to verify whether the effects of preventing the onset and suppressing the progression of retinitis pigmentosa and other retinal degenerative diseases could be achieved.

### (Methods)

### (Animals)

P23H mice (Rho^{P23H/+}), which have been established as a model for retinitis pigmentosa, were used. Specifically, Rho^{P23H/P23H} mice purchased from Jackson Laboratory (Bar Harbor, Me., USA) were mated with C57BL/6J (purchased from Japan Claire Co., Ltd.) to produce the models. The models are suitable mice for observing the suppression of progression of retinitis pigmentosa.

### (Vector Administration)

Zero to three days after birth, AAV DJ-CAGGS-Chimeric rhodopsin (GR/BvRh)-WPRE-pA vector (where the amino acid sequence of chimeric rhodopsin is SEQ ID NO: 1, and the base sequence is represented by SEQ ID NO: 10, etc.) was administered by subretinal injection at a concentration of 1.0×10⁹ vg/μl (1.0×10¹¹ vg/μl in human equivalent) in an amount of 0.5 μl. The same amount of AAV DJ-CAGGS-EGFP-WPRE-pA vector was administered to the control group.

At the age of 24 days and 31 days, retinal tomographic images were taken using an SD-OCT (spectral domain-optical coherence tomography) system (Envisu R4310; Leica, Wetzlar, Germany). The mice were sedated with three types of mixed anesthesia (Midazolam, medetomidine, and butorphanol tartrate were administered at 4 mg/kg and 0.75 mg/kg and 5 mg/kg body weight, respectively). The thickness of the retinal photoreceptor cell layer 150 μm from the optic nerve head was measured in four directions on the upper, lower, ear and nose sides, followed by comparison.

Similarly, at the age of 30 days and 42 days, Measurements were made using electroretinograms.

After more than 8 hours of dark adaptation, the mice were sedated with three types of mixed anesthesia (midazolam, medetomidine, and butorphanol tartrate were administered at 4 mg/kg and 0.75 mg/kg and 5 mg/kg body weight, respectively). The stimulus was measured, using White LEDs, in three stages: rod response (0.01 cd·s·m⁻²), mixed response (3.0 cd·s·m⁻²), and cone response (3.0 cd·s·m⁻²). (Each n=6). As a measuring device, a PuREC acquisition system (Mayo, Inazawa, Japan) was used.

### (Results)

### OCT Results:

At the age of 24 days, there was no significant difference between the control group (49.6±12.4 μm) and the treatment group (61.25±4.44 μm), but a tendency was observed (FIG. 1). At the age of 31 days, the photoreceptor layer was significantly maintained in the mice into which the chimeric rhodopsin gene was introduced (50.7±2.87 μm) compared to the control group (31.8±5.15 μm) (FIG. 1, upper panel).

### ERG Results:

At the age of 30 days, in all the mice, chimeric rhodopsin gene transfer-treated eyes tended to have a large amplitude (FIG. 2, upper left), with the rod response (without treatment: 24.5±13.2 μV, with treatment: 124±43.0 μV), mixed response (without treatment: 24.5±13.2 μV, with treatment: 233±77.3 μV), and cone response (without treatment: 24.5±13.2 μV, with treatment: 176±56.9 μV), where significant differences were observed in the rod response (FIG. 2, lower left, upper right, lower right).

At the age of 42 days, a significant difference in amplitude was observed with all stimuli (FIG. 2, upper left). Furthermore, significant differences were observed in the following: rod response (without treatment: 49.8±16.6 μV, with treatment: 172±19.6 μV); mixed response (without treatment: 118±28.5 μV, with treatment: 295±36.2 μV); cone response (without treatment: 92.6±29.2 μV, with treatment: 258±24.1 μV) (FIG. 2, lower left, upper right, lower right).

### (Discussion)

It is considered that the expression of the chimeric rhodopsin gene exemplary used in the present invention produced the effect of suppressing the progression of retinal degeneration.

It is considered that the fact that the significant differences were observed only in the rod response from the age of 30 days in the result of electroretinogram (upper panel of FIG. 1) may reflect that the disorder is predominantly caused in the rod in retinitis pigmentosa. Furthermore, it is considered that the reason why the amplitude is larger in the treated eyes of the 42 days old, which should be more degenerated than those of the 30 days old, is that the optical response due to the expression of chimeric rhodopsin is added.

In view of the foregoing, the present example demonstrated the potential for the prevention or suppression of the progression of retinal degeneration diseases such as retinitis pigmentosa (particularly before the onset, immediately after the onset, or in the early stage of the onset). Such effects are clinically very significant.

### Example 2: Light-Dark Determination Function Measurement

Next, the effect of the present invention on the light-dark determination function was measured. The descriptions thereof will be provided hereinafter.

### (Materials and Methods)

### (Animals)

Another model of retinitis pigmentosa, rd1 mouse (Pde6b^{rd1/rd1}), was used. A C3H/HeJ Jcl mouse having the above mutation was purchased from Japan Claire Co., Ltd.

### (Vector Administration)

Blind rd1 mice at the age of 10 weeks or older were administered 1 μl of AAV DJ-CAGGS-Chimeric rhodopsin (GR/BvRh)-WPRE-pA vector produced in the preparation example at a concentration of 1.0×10⁹ vg/μl (1.0×10¹¹ vg/μl in human equivalent) by intravitreal injection. The control group was administered the same amount of AAV DJ-CAGGS-EGFP-WPRE-pA vector (vector for expression of EGFP).

### (Measurements)

Measurements were taken at or after the 4th week after the injection, at which gene expression peaked. Mice were placed in a light-dark box (an acrylic case with the width: 415 mm, height: 300 mm, and depth: 250 mm, which is divided into two by a partition, one half of which receives 20 lux of light and the other half of which is a dark room, and the two are connected by a 5×5 mm window) and a video of their 10-minute action was taken. The ratio of staying time in the bright and dark halves was measured and compared. Normally, mice avoid light and they stay in the dark for a long time; however, the retinal degenerated mice had almost the same stay time in the bright and dark halves.

### (Results)

Healthy mice (B6) avoided the bright spot, so that their time spent in the bright spot was shorter (0.137±0.062), while blind mice (rd1) had a staying time ratio of about half (0.48±0.052). In contrast, with the treated mice, a significant reduction in the staying time in the bright spot (0.24±0.049) was observed (FIG. 3).

### (Discussion)

By the treatment conducted in the present example, restoration of the light-dark determination function and the bright spot evading function was observed in the behavioral experiment. It was also found that the behavioral ability to avoid crisis was restored or granted. In view of the foregoing, the present example demonstrated that the present invention improves the light-dark determination function, improves the bright spot evading function and/or improves or restores visual cognitive behavioral functions represented by the crisis avoidance function.

### Example 3: Demonstration of Visual Function Enhancement

Next, the effect of the present invention on the enhancement of the visual functions (e.g., improvement in visual acuity) was measured by the visual evoked potential VEP. The descriptions thereof will be provided hereinafter.

### (Materials and Methods)

### (Animals)

Another model of retinitis pigmentosa, rd1 mouse (Pde6b^{rd1/rd1}), was used. A C3H/HeJ Jcl mouse having the above mutation was purchased from Japan Claire Co., Ltd.

### (Vector Administration)

Blind rd1 mice at the age of 10 weeks or older were administered 1 μl of AAV DJ-CAGGS-Chimeric rhodopsin (GR/BvRh)-WPRE-pA vector at a concentration of 1.0×10⁹ vg/μl (1.0×10¹¹ vg/μl in human equivalent) by intravitreal injection. The control group was administered the same amount of AAV DJ-CAGGS-EGFP-WPRE-pA vector.

### (Measurements)

The visual evoked potential (VEP) was measured at or after the 4th week after the injection, at which the gene expression peaked. One week before the measurement, the mice were subjected to the above-mentioned, three types of mixed anesthesia, and the measurement electrodes were implanted in their skull near the visual cortex (1.5 mm anterior and 1.5 mm lateral to the lambda suture).

After sedation with the three types of mixed anesthesia again, the evoked potential was measured for a flash stimulus of 0.1 cds/m², from a White LED installed 3 cm in front of the eye. As a measuring device, a PuREC acquisition system (Mayo, Inazawa, Japan) was used.

### (Results)

A significant increase in amplitude was observed in the chimeric treated mice (50.0±3.49 μV) with respect to the control (35.12±3.90 μV) (FIG. 4).

### DISCUSSION

A visual sense restoration effect at the central level was also observed as a result of the treatment. It is considered that the present example demonstrated the effect of enhancing the visual functions (improving the visual acuity).

### (Note)

As described above, the present invention has been illustrated using the preferred embodiments of the present invention; however, it is understood that the scope of the present invention should be interpreted only by the Claims thereof. It is understood that the contents of patents, patent applications and documents cited herein should be incorporated herein by reference in the same way that the contents themselves thereof are specifically described herein.

### INDUSTRIAL APPLICABILITY

Pharmaceuticals have been provided for the prevention and the suppression of progression of retinal disease, for the visual cognitive behavioral functions visual cognitive behavioral functions (e.g., improvement in light-dark determination functions, improvement in bright spot evading functions, and/or crisis avoidance functions) and for enhancing the visual acuity. Techniques are provided that are applicable to industries (pharmaceuticals, etc.) based on such techniques as described above.

### SEQUENCE LISTING FREE TEXT

SEQ ID NO: 1: an example of the amino acid sequence of the chimeric rhodopsin according to the present invention
SEQ ID NO: 2: an example of the amino acid sequence of the chimeric rhodopsin according to the present invention
SEQ ID NO: 3: an example of the amino acid sequence of the chimeric rhodopsin according to the present invention
SEQ ID NO: 4: an example of the amino acid sequence of the chimeric rhodopsin according to the present invention
SEQ ID NO: 5: an example of the amino acid sequence of the second loop on the cytoplasmic side of the G protein-coupled receptor rhodopsin according to the present invention.
SEQ ID NO: 6: an example of the amino acid sequence of the second loop on the cytoplasmic side of the G protein-coupled receptor rhodopsin according to the present invention.
SEQ ID NO: 7: an example of the amino acid sequence of the third loop on the cytoplasmic side of the G protein-coupled receptor rhodopsin according to the present invention.
SEQ ID NO: 8: amino acid sequence of Gloeobacter violaceus Rhodopsin (GR)
SEQ ID NO: 9: amino acid sequence of bovine rhodopsin (BvRh)
SEQ ID NO: 10: an example of the base sequence of the chimeric rhodopsin according to the present invention (corresponding to SEQ ID NO: 1), where the start codon corresponds to nucleotides 43-45 and the stop codon corresponds to nucleotides 994-996.
SEQ ID NO: 11: an example of the base sequence corresponding to the second loop on the cytoplasmic side of the G protein-coupled receptor rhodopsin (corresponding to SEQ ID NO: 5)
SEQ ID NO: 12: another example of the base sequence corresponding to the second loop on the cytoplasmic side of the G protein-coupled receptor rhodopsin (corresponding to SEQ ID NO: 6)
SEQ ID NO: 13: an example of the base sequence corresponding to the third loop on the cytoplasmic side of the G protein-coupled receptor rhodopsin
SEQ ID NO: 14: amino acid sequence of human rhodopsin (huRh)
SEQ ID NO: 15: base sequence of human rhodopsin (huRh)
SEQ ID NO: 16: base sequence of bovine rhodopsin (BvRh)
SEQ ID NO: 17: base sequence of Gloeobacter violaceus Rhodopsin (GR)

1. A composition for preventing or suppressing the progression of a disease, disorder or symptom of the retina, the composition comprising a nucleic acid encoding a chimeric protein of an ion-transporting receptor rhodopsin and a G protein-coupled receptor rhodopsin.

2. A composition for improving a visual cognitive behavioral function, the composition comprising a nucleic acid encoding a chimeric protein of an ion-transporting receptor rhodopsin and a G protein-coupled receptor rhodopsin.

3. A composition for enhancing a visual function, the composition comprising a nucleic acid encoding a chimeric protein of an ion-transporting receptor rhodopsin and a G protein-coupled receptor rhodopsin.

4. The composition of claim 1, wherein the disease, disorder or symptom is a retinal degenerative disease.

5. The composition of claim 1 or 4, wherein the disease, disorder or symptom is retinitis pigmentosa.

6. The composition of any one of claims 1, 4 and 5, wherein the disease, disorder or symptom is autosomal dominantly inherited.

7. The composition of any one of claims 1 and 4 to 6, wherein the composition is for preventing or suppressing the progress of retinitis pigmentosa.

8. The composition of any one of claims 1 and 4 to 7, wherein the composition is administered to a subject before the onset or immediately after the onset of the disease, disorder or symptom.

9. The composition of any one of claims 1 to 8, wherein the composition is administered once.

10. The composition of any one of claims 1 to 9, wherein the composition is administered at a unit dose of 0.1×10¹¹ to 10×10¹¹ vg/eye.

11. The composition of any one of claims 1 to 10, wherein, of base sequences encoding the ion-transporting receptor rhodopsin, a base sequence encoding a second loop on a cytoplasmic side and/or a third loop on a cytoplasmic side is substituted by a base sequence encoding a second loop on a cytoplasmic side and/or a third loop on a cytoplasmic side of the G protein-coupled receptor rhodopsin.

12. The composition of any one of claims 1 to 11, wherein the ion-transporting receptor rhodopsin is derived from cyanobacteria (blue-green bacteria).

13. The composition of any one of claims 1 to 12, wherein the G protein-coupled receptor rhodopsin is derived from a mammal.

14. The composition of any one of claims 1 to 13, wherein the chimeric protein is characterized by the amino acid sequence set forth in SEQ ID NO: 8 having a glutamic acid corresponding to position 132 substituted by glutamine.

15. The composition of any one of claims 1 to 14, wherein the chimeric protein is characterized by one or more of:
(a) an amino acid sequence set forth in SEQ ID NOs: 1-4 or a fragment thereof,
(b) an amino acid sequence having at least 80% identity to (a); and
(c) an amino acid sequence with one or more amino acids substituted, added and/or deleted with respect to (a) or (b), and wherein the chimeric protein is further characterized by having biological activity, or wherein the nucleic acid encoding the chimeric protein is characterized by one or more of:
(A) a base sequence encoding an amino acid sequence set forth in any of SEQ ID NOs: 1-4, or a base sequence set forth in SEQ ID NO: 10, or a fragment thereof;
(B) a nucleic acid having at least 80% identity to (A);
(C) a nucleic acid with one or more nucleotides substituted, added and/or deleted with respect to (A) or (B); and
(D) a nucleic acid that hybridizes to any of (A) to (C) under stringent conditions, and the chimeric protein is characterized by having biological activity.

16. The composition of any one of claims 1 to 15, wherein the base sequence is in a vector.

17. A composition for preventing or suppressing the progression of a disease, disorder or symptom of the retina, the composition comprising a chimeric protein of an ion-transporting receptor rhodopsin and a G protein-coupled receptor rhodopsin.

18. A composition for improving a visual cognitive behavioral function, the composition comprising a chimeric protein of an ion-transporting receptor rhodopsin and a G protein-coupled receptor rhodopsin.

19. A composition for enhancing a visual function, the composition comprising a chimeric protein of an ion-transporting receptor rhodopsin and a G protein-coupled receptor rhodopsin.

20. The composition of any one of claims 17 to 19, further having any characteristic in any one or more of claims 4 to 16.

21. A nucleic acid encoding a chimeric protein of an ion-transporting receptor rhodopsin and a G protein-coupled receptor rhodopsin, wherein the chimeric protein is for preventing or suppressing the progression of a disease, disorder or symptom of the retina.

22. A nucleic acid encoding a chimeric protein of an ion-transporting receptor rhodopsin and a G protein-coupled receptor rhodopsin, wherein the chimeric protein is for improving a visual cognitive behavioral function.

23. A nucleic acid encoding a chimeric protein of an ion-transporting receptor rhodopsin and a G protein-coupled receptor rhodopsin, wherein the chimeric protein is for enhancing a visual function.

24. A chimeric protein of an ion-transporting receptor rhodopsin and a G protein-coupled receptor rhodopsin, wherein the chimeric protein is for preventing or suppressing the progression of a disease, disorder or symptom of the retina.

25. A chimeric protein of an ion-transporting receptor rhodopsin and a G protein-coupled receptor rhodopsin, wherein the chimeric protein is for improving a visual cognitive behavioral function.

26. A chimeric protein of an ion-transporting receptor rhodopsin and a G protein-coupled receptor rhodopsin, wherein the chimeric protein is for enhancing a visual function.

27. The nucleic acid or protein of any one of claims 21 to 26, further having any characteristic in any one or more of claims 4 to 16.

28. A method for preventing or suppressing the progression of a disease, disorder or symptom of the retina in a subject in need thereof, the method comprising administering an effective amount of a nucleic acid encoding a chimeric protein of an ion-transporting receptor rhodopsin and a G protein-coupled receptor rhodopsin to the subject.

29. A method for improving a visual cognitive behavioral function in a subject in need thereof, the method comprising administering an effective amount of a nucleic acid encoding a chimeric protein of an ion-transporting receptor rhodopsin and a G protein-coupled receptor rhodopsin to the subject.

30. A method for enhancing a visual function in a subject in need thereof, the method comprising administering an effective amount of a nucleic acid encoding a chimeric protein of an ion-transporting receptor rhodopsin and a G protein-coupled receptor rhodopsin to the subject.

31. A method for preventing or suppressing the progression of a disease, disorder or symptom of the retina in a subject in need thereof, the method comprising administering an effective amount of a chimeric protein of an ion-transporting receptor rhodopsin and a G protein-coupled receptor rhodopsin to the subject.

32. A method for improving a visual cognitive behavioral function in a subject in need thereof, the method comprising administering an effective amount of a chimeric protein of an ion-transporting receptor rhodopsin and a G protein-coupled receptor rhodopsin to the subject.

33. A method for enhancing a visual function in a subject in need thereof, the method comprising administering an effective amount of a chimeric protein of an ion-transporting receptor rhodopsin and a G protein-coupled receptor rhodopsin to the subject.

34. The method of any one of claims 28 to 33, further having any characteristic in any one or more of claims 4 to 16.

35. A pharmaceutical composition comprising a nucleic acid encoding a chimeric protein of an ion-transporting receptor rhodopsin and a G protein-coupled receptor rhodopsin, for preventing or suppressing the progression of a disease, disorder or symptom of the retina.

36. A pharmaceutical composition comprising a nucleic acid encoding a chimeric protein of an ion-transporting receptor rhodopsin and a G protein-coupled receptor rhodopsin, for improving a visual cognitive behavioral function.

37. A pharmaceutical composition comprising a nucleic acid encoding a chimeric protein of an ion-transporting receptor rhodopsin and a G protein-coupled receptor rhodopsin, for enhancing a visual function.

38. A pharmaceutical composition comprising a chimeric protein of an ion-transporting receptor rhodopsin and a G protein-coupled receptor rhodopsin, for preventing or suppressing the progression of a disease, disorder or symptom of the retina.

39. A pharmaceutical composition comprising a chimeric protein of an ion-transporting receptor rhodopsin and a G protein-coupled receptor rhodopsin, for improving a visual cognitive behavioral function.

40. A pharmaceutical composition comprising a chimeric protein of an ion-transporting receptor rhodopsin and a G protein-coupled receptor rhodopsin, for enhancing a visual function.

41. The pharmaceutical composition of any one of claims 35 to 40, further having any characteristic in any one or more of claims 4 to 16.

42. A nucleic acid being characterized by one or more of:
(a) a nucleic acid having an amino acid sequence set forth in SEQ ID NO: 10 or a fragment thereof;
(b) a nucleic acid having at least 80% identity to (a);
(c) a nucleic acid with one or more nucleotides substituted, added and/or deleted with respect to (a) or (b); and/or
(d) a nucleic acid that hybridizes to any of (a) to (c) under stringent conditions, wherein a protein encoded by the nucleic acid is characterized by having biological activity.
